(19) United States
(12) Patent Application Publication
(10) Pub. No.: US 2013/0323673 A1
(43) Pub. Date: Dec. 5, 2013

(54) DENTAL APPARATUS, MEDICAL APPARATUS AND CALCULATION METHOD

(71) Applicant: Sony Corporation, Tokyo (JP)

(72) Inventors: Shiho Hakomori, Kanagawa (JP); Koshi Tamamura, Tokyo (JP)

(73) Assignee: Sony Corporation, Tokyo (JP)

(21) Appl. No.: 13/901,162

(22) Filed: May 23, 2013

(30) Foreign Application Priority Data
Jun. 1, 2012 (JP) 2012125751
Mar. 12, 2013 (JP) 2013048767

(51) Int. Cl. A61B 5/026; A61B 5/0295; A61C 1/00; A61B 5/1455

(52) U.S. Cl. CPC A61B 5/0261; A61B 5/14551; A61B 5/0295; A61C 1/0061; A61B 2576/00
USPC 433/29; 433/215

(57) ABSTRACT

There is provided a dental apparatus including a light source for emitting a light to irradiate at least one of a tooth, a gum, a plaque and a calculus of an oral cavity, a light detector for detecting fluorescence from the oral cavity emitted to the light irradiated from the light source, and a control unit for outputting first data for visualizing a temporal change in a fluorescence intensity based on the fluorescence detected by the light detector. Also, there is provided a calculation method including irradiating an excited light, detecting a fluorescence intensity, and calculating a temporal change in the fluorescence intensity in a depth direction.

### BACKGROUND

The present technology relates to a dental apparatus for use in in periodontitis treatment, diagnosis and the like, and a medical apparatus and a calculation method for use in treatment or prevention of infectious diseases.

### SUMMARY

As periodontitis treatment by a dentist, there are scaling, surgery treatment, treatment by light or ultrasonic and the like. All of which is to remove periodontitis bacteria and calculi, which may beneficially lead to a reduction of inflammation and to a shallow periodontal pocket depth. In addition, aPDT (antimicrobial Photodynamic Therapy) is used for the periodontitis treatment.

In the aPDT treatment, a cationic light-sensitive substance that is itself not toxic and a light having a wavelength for exciting the substance are used. The light-sensitive substance absorbs the light having the wavelength for exciting the substance, becomes in an excited state, and transfers its energy to surrounding oxygen to produce singlet oxygen. The singlet oxygen has high oxidation power, and may damage surrounding cells and tissues. Bacteria have negatively charged surfaces. Therefore, when a drug of the cationic light-sensitive substance is administered on a diseased site, the drug is bonded to the bacteria by an electrostatic interaction. In the state, when the light having the wavelength for exciting the substance is irradiated, the bacteria to which the drug is bonded are killed. For example, Japanese Patent Application Laid-open No. 2011-521237 describes that an effect of PDT in periodontitis treatment is imaged.

It is shown that the aPDT is widely effective for disinfecting infectious microorganisms such as viruses, protozoa, and fungi as well as bacteria (see Masamitsu Tanaka, Pawel Mroz, Tianhong Dail, Manabu Kinoshita, Yuji Morimoto and Michael R. Hamblin, “Photodynamic therapy can induce non-specific protective immunity against a bacterial infection” Proceedings of SPIE Vol. 8224 822403-1). Accordingly, the aPDT is expected to be widely used for treatment and prevention of other infectious diseases in addition to the periodontitis treatment.

In a monitoring apparatus described in Japanese Patent Application Laid-open No. 2011-521237, it is difficult to observe a disinfection progress of the periodontitis treatment using aPDT in real time on a monitor.

It is desirable to provide a dental apparatus and a calculation method capable of observing the disinfection progress of the periodontitis treatment.

According to an embodiment of the present technology, there is provided a dental apparatus including a light source, a light detector, and a control unit.

The light source emits a light for irradiating at least one of a tooth, a gum, a plaque and a calculus of an oral cavity.

The light detector detects fluorescence from the oral cavity emitted to a light irradiated from the light source.

The control unit outputs first data for visualizing a temporal change in a fluorescence intensity based on the fluorescence detected by the light detector.

According to the embodiment of the present technology, by visualizing the temporal change in the fluorescence intensity, a disinfection status of periodontitis bacteria and a removal status of plaques and calculi can be observed almost in real time.

A photosensitizer that is excited by irradiating the light may be distributed in a depth direction of the gum such that the photosensitizer is bonded to or incorporated into periodontitis bacteria, and the control unit may output the first data based on an fluorescence intensity distribution in the depth direction of the gum from the photosensitizer emitted to the light irradiation.

Thus, the distribution of the periodontitis bacteria in the depth direction can be perceived from the image, and the disinfection progress by the treatment of the periodontitis bacteria distributed in the depth direction can be observed almost in real time.

The control unit may calculate a temporal change in the fluorescence intensity in the depth direction based on a calculated temporal change in the distribution of the photosensitizer in a ground state in the depth direction, a calculated temporal change in an intensity distribution of the light in the depth direction, and a fluorescence intensity on a surface of the gum detected by the light detector.

The temporal change in the fluorescence intensity may show a disinfection progress of the periodontitis bacteria.

A photosensitizer that is excited by irradiating the light may be distributed in a depth direction of a plaque or a calculus attached to the tooth or the gum such that the photosensitizer is bonded to or incorporated into periodontitis bacteria, and the control unit may output the first data based on a fluorescence intensity distribution in the depth direction of the gum from the photosensitizer emitted to the light irradiation.

In this way, the distribution of the periodontitis bacteria in the depth direction of the plaques and the calculi attached to the teeth and the gums can be perceived from the image, and the disinfection progress by the treatment of the periodontitis bacteria can be observed almost in real time.

The dental apparatus further includes an image receiving unit for receiving an image of an oral cavity having the tooth and the gum, a positional information receiving unit for receiving positional information about the oral cavity as absolute positional information from a reference position set on an arbitrary position, and an image processing unit for linking image data received at the image receiving unit with positional information received at the positional angle information receiving unit, in which the control unit may correlate a light irradiated site of the oral cavity with the positional information and may output second data for showing the light irradiated site to the image of the oral cavity.

In this way, a patient and a practitioner can observe the disinfection status of the periodontitis bacteria and the removal status of the plaques or the calculi almost in real time, and can perceive a treatment site of the oral cavity.

A photosensitizer is administered into the oral cavity having the tooth and the gum, the photosensitizer being excited by the light irradiation and bonded to or incorporated into periodontitis bacteria, and the control unit may output the first data based on a fluorescence intensity distribution on a surface of the tooth or the gum from the photosensitizer around the surface of the tooth or the gum emitted to the light irradiation.

Thus, the disinfection status of the periodontitis bacteria around the surface of the tooth or the gum or within the plaque can be observed almost in real time.

As the light, a laser light or a light-emitting diode light may be used.

For example, the light is a light having a wavelength belonging, for example, to an absorption band of the photosensitizer.

The light detector may detect at least one of fluorescence, a reflected light and a diffused light from the oral cavity emitted to the light having the wavelength.

The photosensitizer bonded to or incorporated into periodontitis bacteria emits fluorescence by irradiating the light having the wavelength belonging to a specific absorption band such as the red light. Accordingly, the removal status of the plaques and the calculi can be perceived in real time from the temporal change in the fluorescence intensity emitted from the plaques and the calculi.

In addition, the light having the wavelength can be also used as an illumination light source for measuring a blood flow volume or a blood flow speed. Furthermore, the red light can be used as an illumination light source for measuring an oxygen saturation. When the oxygen saturation is measured, the oxygen saturation may be evaluated in combination with an infrared light in some cases. Since the infrared light also has an advantage of encouraging blood circulation, a cure can be encouraged while doing the treatment.

The dental apparatus may further include a blood flow volume detector for detecting a blood flow volume of the gum.

By detecting the blood flow volume, a pain status of the patient can be perceived.

The dental apparatus may further include an oxygen saturation meter for detecting oxygen saturation of the gum.

By detecting the oxygen saturation, a degree of inflammation can be evaluated quantitatively. Also, an effect of PDT (Photodynamic Therapy) can be predicted. In addition, as a basic status of a living body can be perceived, the oxygen saturation becomes a very useful information source to consider the cause of acquiring or not acquiring the PDT effect at a stage of a clinical study.

The dental apparatus may further include an air blowing unit for blowing air to the tooth or the gum.

Since main bacteria engaged in the periodontitis is anaerobes bacteria (strictly anaerobic bacteria or facultative anaerobic bacteria), the diseased site is irradiated with a light while blowing air during the treatment, thereby further improving the disinfection effect.

According to an embodiment of the present technology, there is provided a calculation method including irradiating an excited light, detecting a fluorescence intensity, and calculating a temporal change in the fluorescence intensity in a depth direction.

In irradiating the excited light, a gum of an oral cavity into which a photosensitize is administered is irradiated with an excited light to the photosensitizer.

In detecting the fluorescence intensity, the fluorescence intensity on a surface of a gum id detected.

The temporal change in the fluorescence intensity in the depth direction is calculated based on a calculated temporal change in a distribution of the photosensitizer in the ground state to the depth direction of the gum, a calculated temporal change in the intensity distribution of the excited light in the depth direction, and the fluorescence intensity on the surface of the gum detected.

According to another embodiment of the present technology, there is provided a medical apparatus including a light source, a light detector and a control unit.

The light source emits a light to a treatment site where at least one of treatment and prevention of infectious diseases is implemented.

The light detector detects fluorescence from the treatment site emitted to the light irradiated from the light source.

The control unit outputs data for visualizing a temporal change in a fluorescence intensity based on the fluorescence detected by the light detector.

According to the embodiment of the present technology, by visualizing the temporal change in the fluorescence intensity, a disinfection status of infectious microorganisms such as bacteria and a status of immune system activation can be observed almost in real time.

Specifically, the treatment site is at least one of joint synovium, an abdominal cavity, a choledoch, a tooth root and a salivary gland.

Thus, the above-described medical apparatus can be used for the treatment and the prevention of the infectious diseases under an arthroscopic surgery or laparoscopic surgery, or upon other treatment.

The temporal change in the fluorescence intensity may show a disinfection progress of infectious microorganisms at the treatment site.

In the treatment site, a photosensitizer that is excited by irradiating the light irradiation is distributed.

The control unit may output the data based on a fluorescence intensity distribution at the treatment site from the photosensitizer emitted to the light irradiation.

Thus, a status of a treatment progress can be perceived from the image, and can be observed in real time.

The dental apparatus may further include a blood flow volume detector for detecting a blood flow volume of the treatment site.

A calculation method according to other embodiment includes administering a photosensitizer to a treatment site where at least one of treatment and prevention of infectious diseases is implemented.

The treatment site is irradiated with an excited light to the photosensitizer.

A fluorescence intensity is detected at the treatment site.

A temporal change in the fluorescence intensity at the treatment site based on the fluorescence intensity at the treatment site from the photosensitizer emitted to the light irradiation is calculated.

According to the embodiment of the present technology, a disinfection status of periodontitis bacteria and a removal status of plaques and calculi can be observed almost in real time.

### BRIEF DESCRIPTION OF DRAWINGS

FIG. 1 shows a schematic configuration diagram of a dental apparatus according to a first embodiment of the present technology;

FIG. 2 is a functional block diagram of the dental apparatus shown in FIG. 1;

FIG. 3 shows that an oral cavity is image-captured using a three-dimensional model acquiring probe configuring a part of the dental apparatus shown in FIG. 1;

FIG. 4 is a front view of a tip of the three-dimensional model acquiring probe of the dental apparatus shown in FIG. 1;

FIG. 5 is a rear view of a tip of the three-dimensional model acquiring probe of the dental apparatus shown in FIG. 1;

FIG. 6 is a schematic configuration diagram of a light-field camera;

FIGS. 7A and 7B each shows an image example of the three-dimensional model of an oral cavity generated using the three-dimensional model acquiring probe shown in FIG. 4;

FIGS. 8A and 8B each shows periodontitis treatment using a probe for treatment and diagnosis configuring a part of the dental apparatus shown in FIG. 1;

FIG. 9 shows a tip surface of the probe for treatment and diagnosis of the dental apparatus shown in FIG. 1;

FIG. 10 is a schematic sectional diagram by cutting the probe for treatment and diagnosis shown in FIG. 9 along a line B-B′, and shows that periodontitis bacteria in a gum are disinfected using the treatment probe;

FIG. 11 is a flow diagram showing a flow of diagnosis and treatment of periodontitis using the dental apparatus shown in FIG. 1;

FIG. 12 is a flow diagram showing a flow of a processing of acquiring the three-dimensional model by the dental apparatus shown in FIG. 1;

FIG. 13 is a flow diagram showing a flow of a processing of acquiring an image showing a disinfection effect of a gum in a depth direction upon the treatment by the dental apparatus shown in FIG. 1;

FIGS. 14A to 14C are each a graph for illustrating a method of estimating the disinfection effect from an attenuation amount in a fluorescence intensity when the image showing the disinfection effect of a gum or the plaques in the depth direction shown in FIG. 13 is acquired;

FIG. 14D is a graph for visualizing the disinfection effect;

FIG. 15 is a graph for illustrating a method of finding the graph shown in FIG. 14B;

FIG. 16 is a diagram for illustrating the PDT reaction;

FIG. 17 is a configuration diagram for showing a reference light type Doppler meter for calculating a blood flow volume in the dental apparatus shown in FIG. 1;

FIG. 18 is a configuration diagram for showing a differential type Doppler meter for calculating a blood flow volume in the dental apparatus shown in FIG. 1;

FIG. 19 shows an example of an image displayed on a monitor of the dental apparatus shown in FIG. 1;

FIG. 20 is a diagram for showing another example of the probe for treatment and diagnosis;

FIG. 21 is a diagram for showing a still another example of the probe for treatment and diagnosis;

FIG. 22 is a diagram for showing a still another example of the probe for treatment and diagnosis;

FIG. 23 is a diagram for showing a still another example of the probe for treatment and diagnosis;

FIG. 24 is a diagram for showing a still another example of the probe for treatment and diagnosis;

FIG. 25 is a diagram for showing a still another example of the probe for treatment and diagnosis;

FIG. 26 is a diagram for showing a still another example of the probe for treatment and diagnosis;

FIG. 27 is a flow diagram showing a flow of a processing of acquiring an image showing a disinfection effect of a gum in a depth direction upon the treatment by the dental apparatus shown in FIG. 1 according to a second embodiment;

FIGS. 28A to 28C are each a diagram for illustrating a method of estimating the disinfection effect from an attenuation amount in a fluorescence intensity when the image showing the disinfection effect of a gum or the plaques in the depth direction shown in FIG. 27 is acquired;

FIG. 28D is a graph for visualizing the disinfection effect;

FIG. 29 shows a functional block diagram of the dental apparatus according to a third embodiment of the present technology;

FIG. 30 is an overall view of a probe for treatment and diagnosis configuring a part of the dental apparatus shown in FIG. 29;

FIG. 31 shows a tip surface of the probe for treatment and diagnosis of the dental apparatus shown in FIG. 30;

FIG. 32 shows treatment or diagnosis by the probe for treatment and diagnosis shown in FIG. 30;

FIG. 33 is a flow diagram of treatment and diagnosis using the dental apparatus shown in FIG. 29;

FIG. 34 shows an example of an image displayed on a monitor of the dental apparatus shown in FIG. 1;

FIG. 35 is an overall view of a probe for treatment and diagnosis according to a fourth embodiment;

FIG. 36 shows a tip surface of the probe for treatment and diagnosis of the dental apparatus shown in FIG. 35;

FIG. 37 shows treatment by the probe for treatment and diagnosis shown in FIG. 35;

FIG. 38 is a graph showing a relationship between light reachability of a polychromatic light and absorption coefficient of a photosensitizer;

FIG. 39 is a schematic diagram showing an arthroscopic surgery;

FIG. 40 shows a schematic configuration diagram of a medical apparatus 1A according to a sixth embodiment;

FIG. 41 is a functional block diagram of the medical apparatus 1A shown in FIG. 40;

FIG. 42 shows an example of an image displayed on the display unit 21A of the monitor 2A;

FIG. 43 shows a flow diagram showing a flow of diagnosis and treatment using the medical apparatus shown in FIG. 40;

FIG. 44 is a diagram showing an implementation of an aPDT using the medical apparatus shown in FIG. 40;

FIG. 45 is a flow diagram showing a flow of steps of implementing the aPDT before a surgery;

FIG. 46 is a schematic sectional diagram showing a configuration of a medical apparatus according to an alternative embodiment of the sixth embodiment;

FIG. 47 is a schematic sectional diagram of a tip of a treatment probe according to the alternative embodiment of the sixth embodiment;

FIG. 48 is a schematic diagram showing a laparoscopic surgery;

FIG. 49 shows a flow diagram showing a flow of diagnosis and treatment using a medical apparatus according to a seventh embodiment;

FIG. 50 is a diagram showing an implementation of an aPDT using the medical apparatus shown in FIG. 49;

FIG. 51A is a diagram showing an implementation of an aPDT using a medical apparatus according to an alternative embodiment of the seventh embodiment;

FIG. 51B is a diagram showing an implementation of an aPDT using a medical apparatus according to an alternative embodiment of the seventh embodiment;

FIG. 52 is a diagram showing an implementation of an aPDT using a reflection apparatus according to an alternative embodiment of the reflection unit shown in FIG. 51A or 51B;

FIG. 53 is a plan diagram showing a tip surface of a treatment probe according to an alternative embodiment of the seventh embodiment;

FIG. 54 is a schematic diagram showing a treatment of choledocholithiasis; and

FIG. 55 is a plan view showing a configuration of a tip of an endoscope of a medical apparatus according to eighth embodiment.

### DETAILED DESCRIPTION OF EMBODIMENTS

Hereinafter, an embodiment of the present technology will be described with reference to the drawings.

### Summary of Embodiments

The embodiments of the present technology relate to a dental apparatus for use in periodontitis treatment and diagnosis and removal of plaques and calculi.

In periodontitis treatment and removal of plaques and calculi by a dentist, perceiving the effects by a patient motivates to do positive treatment and care.

According to the embodiment of the present technology, the oral cavity is irradiated with the light, and the temporal change in the fluorescence intensity or the reflected light from the oral cavity emitted to the light irradiated is visualized. In this way, the disinfection status of periodontitis bacteria and the removal status of plaques and calculi can be observed in real time. The patient can realize the effects of the treatment and the care.

When disinfection treatment and the disinfection status is observed, Photodynamic Therapy (hereinafter referred to as “PDT”) using the photosensitizer and an excitation light for exciting the photosensitizer can be used.

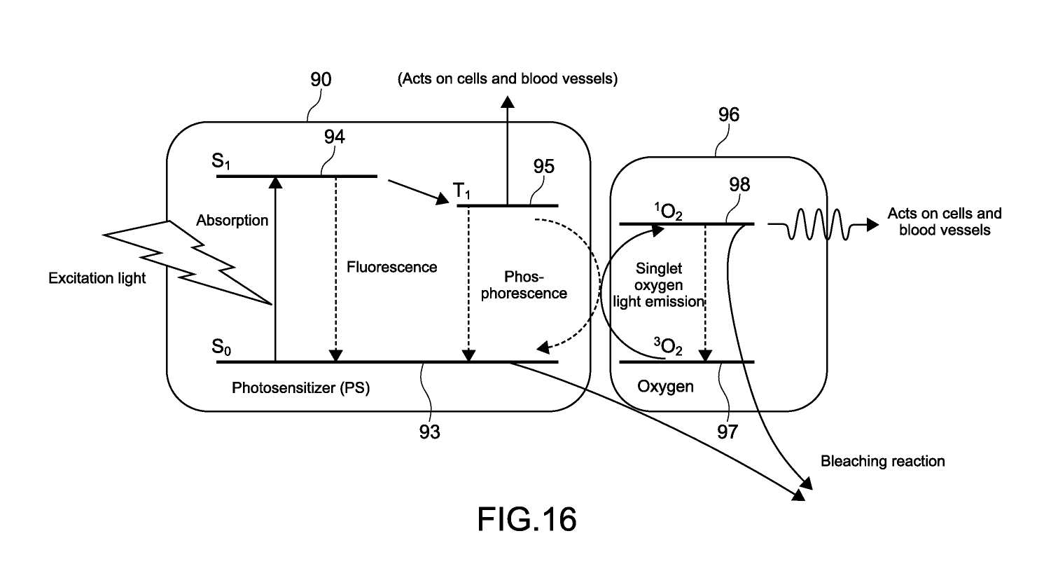

FIG. 16 shows a photochemical reaction of the PDT.

As shown in FIG. 16, a photosensitizer 90 absorbs the excitation light, receives energy and is changed from a ground state 93 to a singlet excited state 94. Most energy is transferred by an intersystem crossing from the singlet excited state 94 to a triplet excited state 95. A part of remaining energy returns from the singlet excited state 94 to the ground state 93. At this point, fluorescence is emitted. In addition, when the photosensitizer 90 in the triplet excited state 95 is collided with oxygen 97 in a triplet state, the energy is transferred to oxygen, and singlet oxygen 98 having high oxidation power is produced. The oxidation power damages surrounding cells and tissues, and destroys (breaches) the photosensitizer 90. By breaching, an amount of the sensitizer to be effective is decreased, and an amount of fluorescence is also decreased. Thus, a decrease in the fluorescence amount forms an indicator of the bleaching and an amount of the tissues damaged.

Here, surfaces of the periodontitis bacteria are negatively charged. When a cation photosensitizer is administered, the photosensitizer is bonded to the periodontitis bacteria by an electrostatic interaction. Under the state, the excitation light is irradiated for exciting the photosensitizer to kill only the periodontitis bacteria bonded to the photosensitizer. By administrating a photosensitizer that will be incorporated into the periodontitis bacteria, the periodontitis bacteria in which the photosensitizer is incorporated are killed once the excitation light is irradiated.

Thus, by administering the photosensitizer into the oral cavity and irradiating the excitation light thereto, the periodontitis bacteria can be disinfected, and the removal status of the periodontitis bacteria can be perceived by the temporal change in the fluorescence intensity emitted from the oral cavity.

As the excitation light of the photosensitizer, a laser light, a light-emitting diode light or a white light source can be used.

Further, according to the embodiment, periodontitis bacteria entered into a gum distributed in a depth direction or periodontitis bacteria in plaques or calculi can be observed for a temporal change in a disinfection status in a depth direction.

The temporal change in the disinfection status in the depth direction can be calculated based on a calculated temporal change in a distribution of a photosensitizer in the ground state in a depth direction, a calculated temporal change in an intensity distribution of light in a depth direction, and a fluorescence intensity on gum surfaces detected by a light detector.

A temporal change in a disinfection status of periodontitis bacteria that are present on teeth or gum surfaces, i.e., are two-dimensionally present can be observed.

Instead of the photosensitizer, the oral cavity is irradiated with blue light etc. From the fluorescence intensity emitted from the plaques and the calculi attached to the teeth and the gums, the removal status of the plaques and the calculi on the tooth and gum surfaces can be perceived.

By irradiating blue light, the plaques and calculi emit fluorescence. Utilizing this, the temporal change in the fluorescence intensity is observed upon the removal of the plaques and calculi, thereby observing in real time the removal status of the plaques and the calculi.

The image of the oral cavity may be captured before treatment. In this case, the reference position is set on an arbitrary position, positional information about teeth, gums and the like of the oral cavity is acquired as absolute positional information from the reference position, and the positional information is linked to the image data. In addition, by correlating an excitation light irradiated site of the oral cavity upon the treatment to the positional information, there can be provided data showing the position of the oral cavity irradiated with the excitation light. In this way, a display unit can display the image showing the treatment site of the oral cavity upon the treatment. By observing the image, the treatment site can be perceived.

Hereinafter, a first embodiment according to the present technology will be described below referring to Figures.

### First Embodiment

The first embodiment illustrates that a photosensitizer and an excitation light having an absorption wavelength of the photosensitizer are used to treat or diagnose periodontitis.

According to the first embodiment, a three-dimensional model of an oral cavity having teeth and gums is firstly acquired. Next, the photosensitizer is administered into the oral cavity, the excitation light is irradiated, and treatment or diagnosis is done. Upon the treatment, an image of the site of the oral cavity on which the excitation light is irradiated, i.e., treated is displayed on the three-dimensionally model acquired, and an image of a disinfection status of periodontitis bacteria distributed to the gums, the plaques or the calculi in a depth direction is also displayed on a display unit of a monitor.

A configuration of a dental apparatus in the first embodiment will be described.

### 1. Configuration of Dental Apparatus

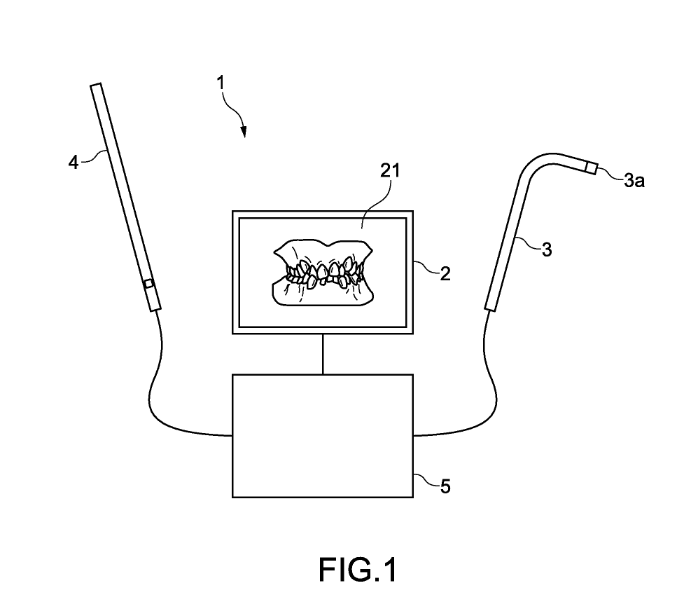

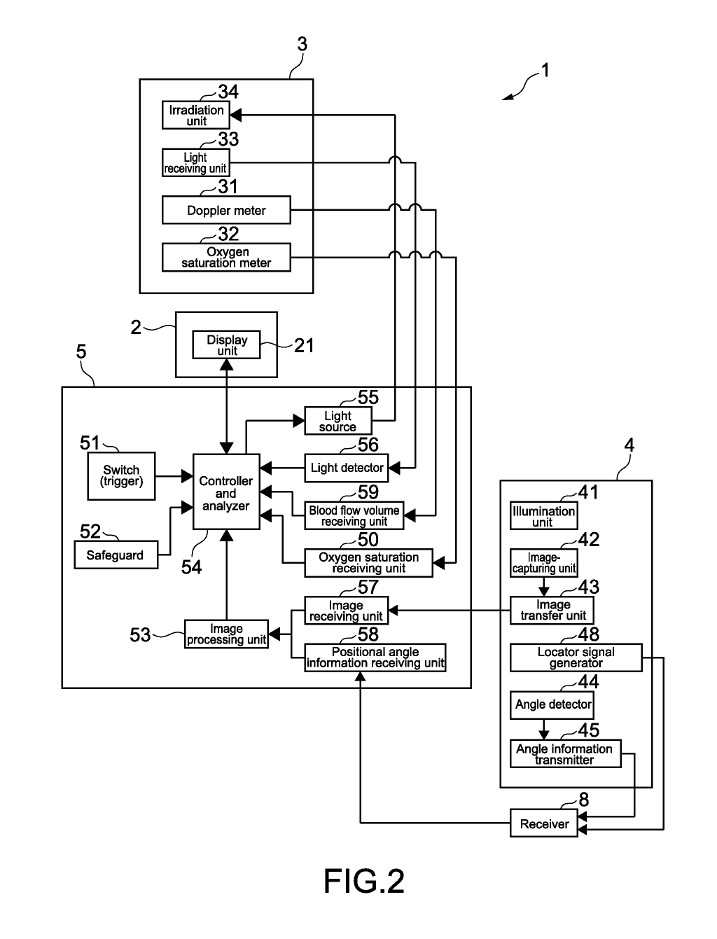

FIG. 1 shows a schematic configuration diagram of a dental apparatus 1 according to the first embodiment. FIG. 2 is a functional block diagram of the dental apparatus shown in FIG. 1.

As shown in FIGS. 1 and 2, the dental apparatus 1 includes a monitor 2, a main unit 5, a three-dimensional model acquiring probe 4, a probe for treatment and diagnosis 3 and a receiver 8. In FIG. 1, the receiver 8 is not shown.

### 1.1 Configuration of Monitor

The monitor 2 is a display apparatus having a display unit 21 displaying an image. The monitor 2 is connected wired or wireless to the main unit 5. Alternatively, the monitor 2 may not be disposed, and the main unit 5 may include the display unit.

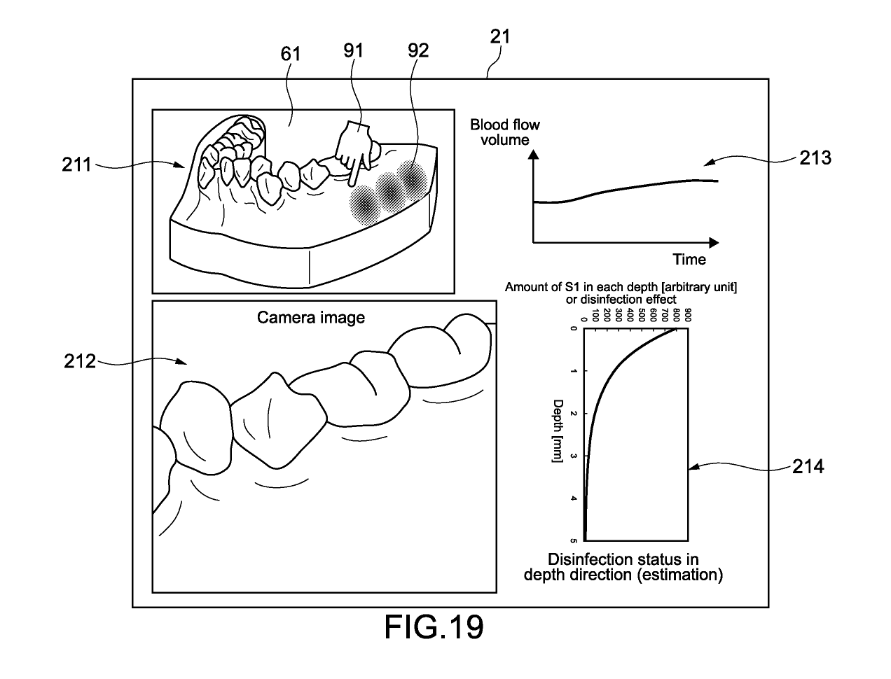

FIG. 19 shows an example of an image displayed on the display unit 21 of the monitor 2.

At a left upper area in the display unit 21, a three-dimensionally model 211 of the oral cavity acquired by using the three-dimensional model acquiring probe 4 is displayed. On the three-dimensional model 211, there is displayed a finger arrow mark 91 that points a site being treated by irradiating a treatment and diagnosis light. Also, sites already treated are enclosed by circles 92, whereby a treatment history can be perceived. In addition, a treatment effect magnitude can be visually mapped.

At a left lower area in the display unit 21, an actual image (a camera image) 212 of sites being treated by a probe for treatment and diagnosis 3 is displayed. The actual image 212 is captured by a capturing unit disposed on the probe for treatment and diagnosis 3, for example.

At a right upper area on the display unit 21, a graph image 213 is displayed. The graph image 213 shows a temporal change in the blood flow volume determined by a Doppler meter 31 of the probe for treatment and diagnosis 3.

At a right lower area on the display unit 21, a graph image 214 is displayed. The graph image 214 shows a disinfection status of periodontitis bacteria of the gums in a depth direction or a temporal change in an amount of the photosensitizer in the ground state. In the image 214, S1 denotes the photosensitizer 94 in the ground state.

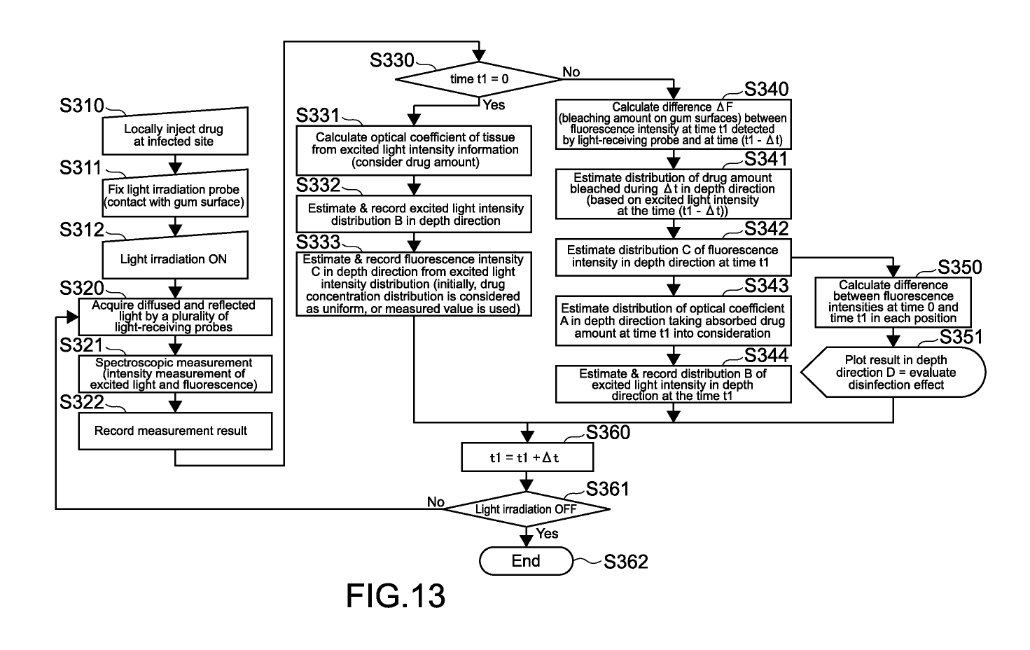

An ordinate axis of the image 214 may be an indicator of a disinfection effect calculated in accordance with a flow diagram shown in FIG. 13.

### 1.2. Configuration of Receiver

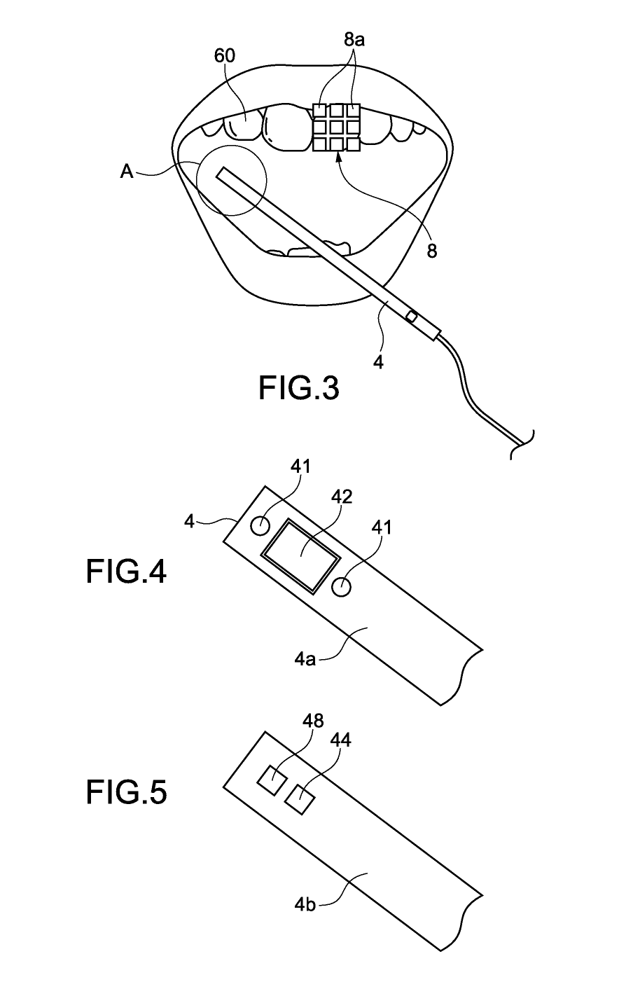

FIG. 3 shows that an oral cavity is image-captured using a three-dimensional model acquiring probe 4.

As shown in FIGS. 2 and 3, the receiver 8 or a spatial angle detector is a magnetic sensor group having a number of magnetic sensors 8a is arranged in a mesh.

The receiver 8 is disposed on one tooth arbitrary selected, for example, the left upper central incisor, upon the image-capturing and the periodontitis treatment.

Upon the image-capturing and the treatment, a receiver 8 is disposed at the same site of the oral cavity.

The receiver 8 will form the reference position when the positional information about the image captured site is acquired upon the image-capturing. The receiver 8 receives a locator signal from a locator signal generator 48 as described later, determines the positional information of the site captured as absolute positional information from the receiver 8 as the reference position, and transmits it to a positional angle information receiving unit 58 as described later concerning the main unit 5.

### 1.3. Configuration of Three-Dimensional Model Acquiring Probe

As shown in FIG. 3, the three-dimensional model acquiring probe 4 image-captures the oral cavity. The three-dimensional model acquiring probe 4 is a stick having a gripper. A practitioner grips it, enters a tip of the three-dimensional model acquiring probe 4 into the oral cavity to image-capture the teeth and the gums. Upon the image-capturing, the receiver 8 is disposed on one tooth arbitrary selected, for example, the left upper central incisor.

Upon the image-capturing, the oral cavity is partly image-captured. The three-dimensional model acquiring probe 4 scans the oral cavity and image-captures to acquire a plurality of partial image data. The main unit 5 described later constructs the plurality of partial image data to form a three-dimensional model of the oral cavity.

The three-dimensional model acquiring probe 4 is connected wired or wireless to the main unit 5.

As shown in FIG. 2, the three-dimensional model acquiring probe 4 includes an illumination unit 41, an image-capturing unit 42, an image transfer unit 43, a locator signal generator 48, an angle detector 44 and an angle information transmitter 45.

The illumination unit 41 emits a light for illuminating the site to be image-captured in the oral cavity upon the image-capturing.

The image-capturing unit 42 converts the image data of the light in the oral cavity incident on a lens into an electrical signal.

The image transfer unit 43 transfers the image data acquired in the image-capturing unit 42 to an image receiving unit 57 of the main unit 5.

The locator signal generator 48 transmits the locator signal being the positional information of the site image-captured to the receiver 8.

The angle detector 44 detects an image-capturing angle of the three-dimensional model acquiring probe 4 upon the image-capturing using an accelerator sensor, an MEMS gyro sensor and the like.

The angle information transmitter 45 transmits image-capturing angle information of the three-dimensional model acquiring probe 4 received from the angle detector 44 to the positional angle information receiving unit 58 of the main unit 5, and links the image data with the positional information received via the receiver 8 at the image processing unit 53 of the main unit 5.

FIG. 4 is a schematic front view of a tip of the three-dimensional model acquiring probe 4.

As shown in FIG. 4, on a front 4a of the tip of the three-dimensional model acquiring probe 4, there are disposed the illumination unit 41 for illuminating the light to the oral cavity, and a CMOS (Complementary Metal Oxide Semiconductor) image sensor having an array lens 42 that is the image-capturing unit.

The CMOS image sensor having an array lens 42 is an image-capturing device where the teeth and the gums to be image-captured are illuminated with the light emitted from the illumination unit 41, a returned light reflected by the teeth and the gums is imaged on a light-receiving surface of the image sensor, and light and dark parts of the image by the light are photoelectrically converted to a charge amount and are read out to convert it into an electrical signal.

The CMOS image sensor having an array lens 42 includes an array lens having different focal points. The CMOS image sensor may be a CCD image sensor or the like.

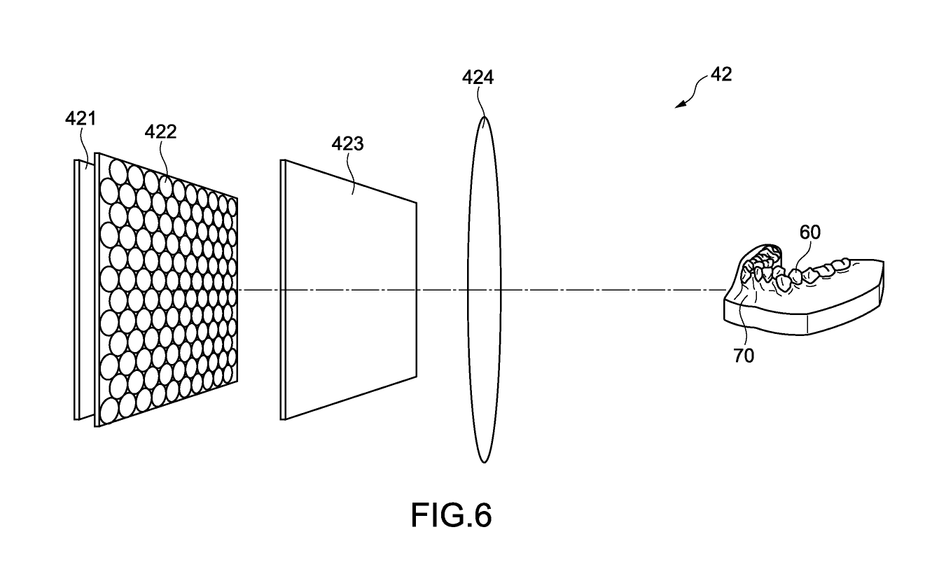

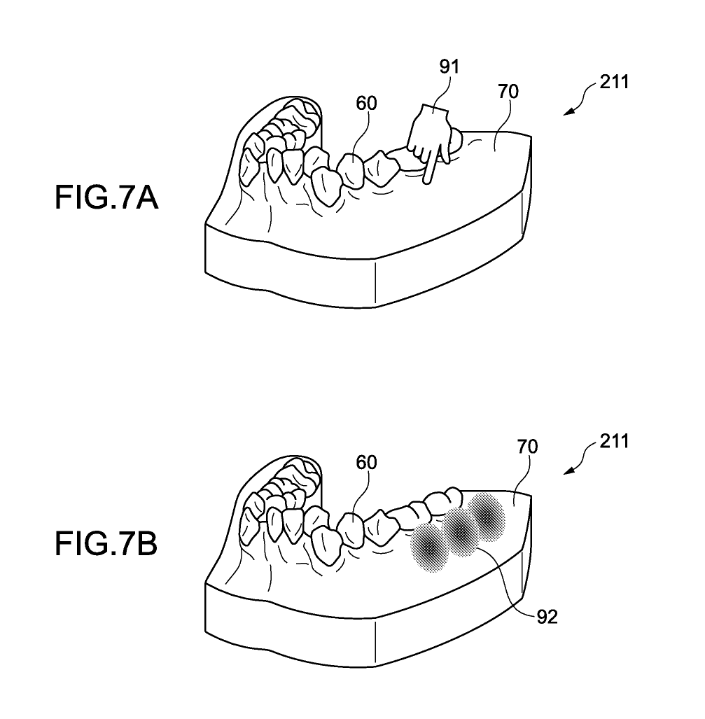

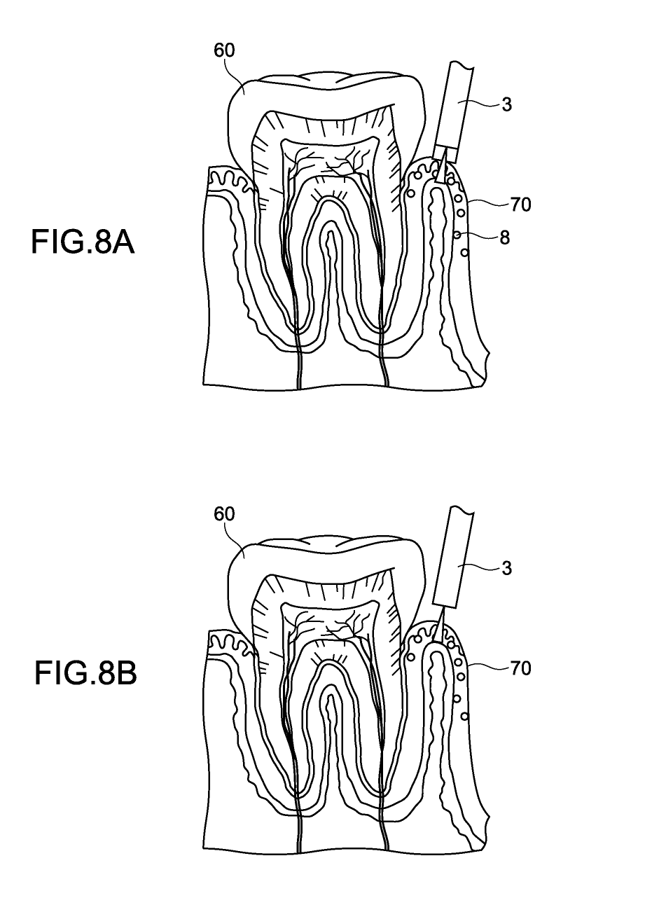

FIG. 6 is a schematic diagram of the CMOS image sensor having an array lens 42. FIG. 7A shows an example of an image of three-dimensional model of the oral cavity that is image-captured by the probe for acquiring the three-dimensional model 4, processed at the main unit 5 and displayed on the display unit 21.

The CMOS image sensor having an array lens 42 includes a main lens 424, a reconstruction image plane 423, an array lens 422, and an image plane 421.

The array lens 422 has different focal points. The focal points are reconstructed, whereby various focused images can be provided without changing focuses of the main lens 424. By using the image sensor having the array lens, images having different focus positions can be provided at one time image-capturing. Also, the images acquired at respective focal lengths are processed, whereby all focused images can be constructed and depth information can be provided. In this way, the three-dimensional model 211 can be provided as the image in the oral cavity, as shown in FIG. 7A.

In the first embodiment, the image of the oral cavity is acquired by the CMOS image sensor having an array lens 42. Accordingly, there is no radiation-exposure like x-rays, and a stereoscopic arrangement in the oral cavity having the gums can be reproduced as the three-dimensional model. In addition, stereoimages can be readily provided without waiting until a molded product, i.e., a denture mold, is completed. Furthermore, the gums that are not imaged by x-rays can be visualized stereoscopically.

Also, the CMOS image sensor having an array lens 42 may have a mechanism for warming the sensor 42 to the similar temperature of the oral cavity so that the sensor 42 is not fogged up.

FIG. 5 is a schematic diagram of a rear of a tip of the three-dimensional model acquiring probe 4.

As shown in FIG. 5, a rear 4b of the tip of the three-dimensional model acquiring probe 4 includes the locator signal generator 48 for generating a locator signal, an acceleration sensor or an MEMS (Micro Electro Mechanical Systems) gyro sensor 44.

As the locator signal generator 48, a magnetism generator or the like is used. The locator signal generator 48 generates a locator signal.

The acceleration sensor or the MEMS gyro sensor 44 is an angle detector for detecting image-capturing angle information of the three-dimensional model acquiring probe 4 upon the image-capturing.

### 1.4. Configuration of Probe for Treatment and Diagnosis

Returning to FIG. 1, the probe for treatment and diagnosis 3 is a stick having a gripper.

FIG. 8A shows that a tip of the probe for treatment and diagnosis 3 is contacted with a diseased site to treat and diagnose the site. FIG. 8B shows that the tip thereof is not contacted with the diseased site to treat the site.

As shown in FIGS. 8A and 8B, the probe for treatment and diagnosis 3 is inserted into the oral cavity upon the treatment and the diagnosis such that the tip thereof is contacted or not contacted with the diseased site. The probe for treatment and diagnosis 3 emits a laser light, an LED light or the like that excites the photosensitizer for killing the periodontitis bacteria.

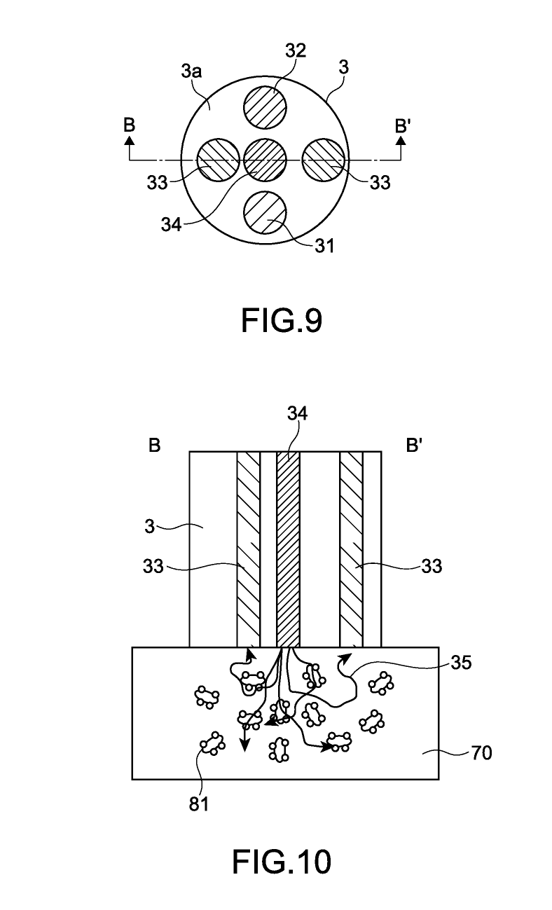

FIG. 9 is a diagram of a tip surface of the probe for treatment and diagnosis 3.

FIG. 10 is a schematic sectional diagram of the tip of the probe for treatment and diagnosis 3.

As shown in FIGS. 2, 9 and 10, the probe for treatment and diagnosis 3 includes a light-receiving probe 33 that is a light receiving unit, a light irradiation probe for treatment and diagnosis 34 that is an irradiation unit, a Doppler meter 31, and an oxygen saturation meter 32.

The light irradiation probe for treatment and diagnosis 34 includes fibers guiding a laser light and an LED light for treatment each having a wavelength for exciting the photosensitizer emitted from a light source 55 of the main unit 5 as described later. The laser light and the LED light emitted from the light source 55 guided by the fibers are emitted. As shown in FIG. 10, the irradiated light 35 emitted from the light irradiation probe for treatment and diagnosis 34 sterilizes the periodontitis bacteria 81 bonded to the photosensitizer distributed in the gum 70. The plaques or the calculi may be irradiated with the irradiated light 35 to sterilize the periodontitis bacteria 81 bonded to the photosensitizer.

The light-receiving probe 33 includes fibers that guide fluorescence and diffused and reflected light that are emitted from the oral cavity to the irradiated light.

The irradiated light is emitted from the light irradiation probe for treatment and diagnosis 34 to the diseased site.

The fluorescence and the diffused and reflected light received at the light-receiving probe 33 are guided to the main unit 5 by optical fibers, and split at a light detector 56 of the main unit 5 as described later. Then, fluorescence intensity and diffused and reflected light intensity are detected.

A plurality of the light-receiving probes 33 is disposed. In the first embodiment, two light-receiving probes 33 are disposed. When the plurality of the light-receiving probes 33 is disposed, the light-receiving probes are disposed such that lengths from the light-receiving probes to the light irradiation probe for treatment and diagnosis 34 are changed. Alternatively, the imaging fibers may be used as the light-receiving probe 33, thereby detecting by the imaging fibers alone.

The Doppler meter 31 measures the blood flow volume of blood vessels at the diseased site with which the light for treatment and diagnosis is irradiated, for example, using the light having a wavelength of 633 nm. The blood flow volume is calculated using a Doppler shift.

Also, the blood flow volume may be determined by Fourier transformation of a speckle pattern of the reflected light.

The Doppler meter 31 transmits the information about the blood flow volume measured to a blood flow volume receiving unit 59 of the main unit 5.

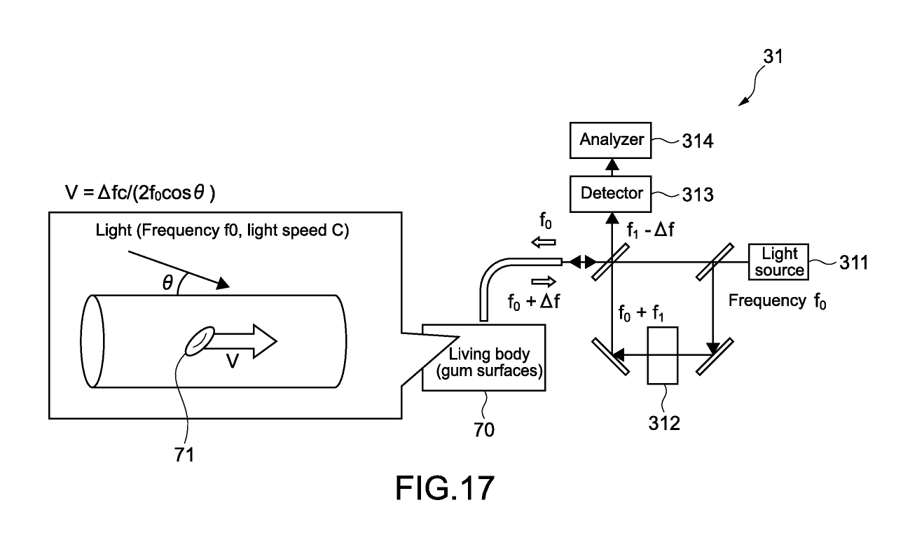

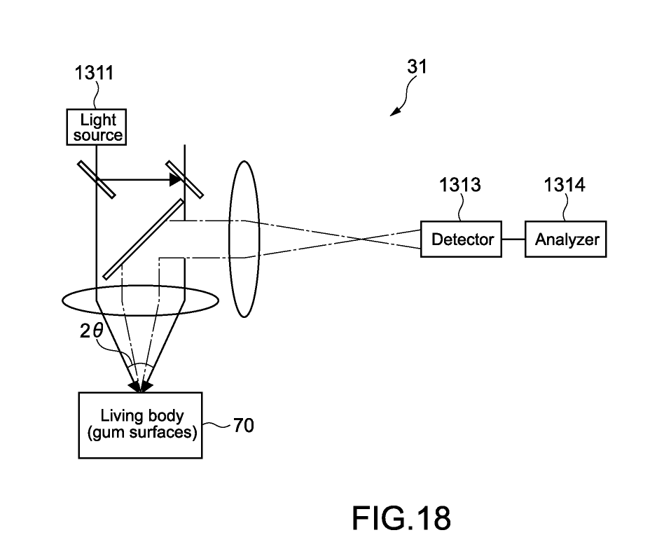

FIG. 17 shows a measurement system using a reference light type Doppler shift. FIG. 18 shows a measurement system using a differential type Doppler shift.

As shown in FIG. 17, a reference light type Doppler meter 31 includes a light source 311, a frequency shifter 312, a detector 313 and an analyzer 314.

In the Doppler meter 31, a migration speed of erythrocytes is detected as a blood flow speed. In the Doppler meter 31, the gum surfaces 70 are irradiated with a laser light having a frequency f₀ outputted from the light source 311. The laser light is scattered to erythrocytes 71 of the blood that move at a speed V. The frequency of the scattered light is slightly shifted for the migration speed of the erythrocytes by the Doppler effect (Doppler shift), and becomes f₀+Δf. The detector 313 detects the frequency of the scattered light.

The analyzer 314 detects the Doppler shift in the scattered light using the light before being incident in the gum 70 as a reference light. Based on the Doppler shift, the speed v is determined. The frequency shifter 312 is disposed to distinguish a displacement direction of the erythrocytes 71 to be measured. The analyzer 314 distinguishes the displacement direction of the subject to be measured.

As shown in FIG. 18, a differential type Doppler meter 1031 includes a light source 1311, a detector 1313 and an analyzer 1314.

In the differential type, one laser beam from the light source 1311 is split into two laser beams. The two beams are collected and crossed. At a crossed position, interference of the scattered light is generated by laser light irradiation directions. Spaces between interference stripes are different due to the Doppler shift amounts of the erythrocytes in the gum 70 to be measured, and are detected by the detector 1313. The analyzer 1314 determines the blood flow speed.

As described above, a component of the blood flow speed can be determined by the change from the frequency of the irradiated light. By calculating an amount of light in a modulation component, erythrocyte components corresponding to the blood flow can be determined.

The oxygen saturation meter 32 measures oxygen saturation at the diseased site with which the treatment and diagnosis light is irradiated. For example, the oxygen saturation meter 32 measures oxygen saturation in blood using a red light having a wavelength of about 665 nm and an infrared light having a wavelength of about 880 nm by utilizing a difference in absorbance of the two lights by oxyhemoglobin and deoxyhemoglobin in blood. By the oxygen saturation, a degree of inflammation can be quantitatively evaluated. Also, an effect of the PDT can be predicted. In addition, by the oxygen saturation, a basic status of a living body can be perceived, which may be a very useful information source to consider a cause of the PDT effect obtained and a cause of the PDT effect not obtained in a clinical study.

An oxygen saturation receiving unit 50 of the main unit 5 receives the oxygen saturation measured in the oxygen saturation meter 32.

A periodontitis patient may feel a pain during the treatment, as the diseased site may have a lower blood flow volume. By disposing the Doppler meter 31 and the oxygen saturation meter 32, a process of relieving the pain can be confirmed and quantitatively monitored by checking the blood flow volume and the oxygen saturation.

The diseased site by the periodontitis may typically swells, the blood flow volume and the oxygen saturation are measured before the treatment and diagnosis light is irradiated, thereby identifying the diseased site.

### 1.5. Configuration of Main Unit

As shown in FIG. 2, the main unit 5 includes a light source 55, a light detector 56, an image receiving unit 57, a positional angle information receiving unit 58, an image processing unit 53, and a controller and analyzer 54.

The main unit 5 includes a switch 51, a safeguard 52, a blood flow volume receiving unit 59, and an oxygen saturation receiving unit 50.

The light source 55 emits a light (excited light) that corresponds to an absorption wavelength of the photosensitizer administered into the oral cavity. In the first embodiment, the light source 55 emits a laser light. The laser light from the light source 55 is emitted from the light irradiation probe for treatment and diagnosis 34 of the probe for treatment and diagnosis 3. Although the light irradiated to the diseased site is the laser light in the first embodiment, a light-emitting diode light or a white light source may be used.

As the treatment and diagnosis light, a red light can be used. The red light can be used as a PDD/PDT light source, a light source for measuring a blood flow, and a light source for measuring oxygen saturation.

The treatment and diagnosis light is not limited to the red light, and may be the light belonging to the absorption band of the photosensitizer.

The light detector 56 splits the light received at the light-receiving probe 33 of the probe for treatment and diagnosis 3, and detects intensity of each of fluorescence and diffused and reflected light.

The image receiving unit 57 receives and records the image data of the oral cavity having the teeth and the gums transmitted from the image transfer unit 43 of the three-dimensional model acquiring probe 4. Also, the image receiving unit 57 can receive and record the image from an imager of the probe for treatment and diagnosis 3.

The positional angle information receiving unit 58 receives and records positional information (spatial positional information and the image-capturing angle information) from the angle information transmitter 45 of the three-dimensional model acquiring probe 4. The positional angle information receiving unit 58 receives image-capturing positional information of the oral cavity as absolute positional information from the reference position set at an arbitrary position, but is set here at the left upper central incisor.

The image processing unit 53 analyzes the image data recorded on the image receiving unit 57 per focal depth, and constructs the all focused images.

The image processing unit 53 links the image data received at the image receiving unit 57 with the positional information received at the positional angle information receiving unit 58.

The controller and analyzer 54 operates by turning on a switch 51.

The controller and analyzer 54 stops the operation of the main unit 5 when the safeguard 52 recognizes that the main unit 5 is under risky working conditions.

The controller and analyzer 54 merges the all focused images, the positional information and the positional angle information linked in each of the plurality of the partial image data of the oral cavity to construct the three-dimensional model of the oral cavity, outputs the data for visualizing the three-dimensional model, and displays the three-dimensional model on the display unit 21.

The controller and analyzer 54 correlates the image data received from the image processing unit 53 to which the positional information is linked to a light irradiation position of the probe for treatment and diagnosis 3, outputs second data for visualizing that a laser-irradiated position or positions of the three-dimensional model of the oral cavity, and displays the three-dimensional model of the oral cavity where the treatment site is shown on the display unit 21.

The light-irradiated position of the oral cavity may be displayed such that a doctor clicks the irradiated position on the three-dimensional model by a finger arrow mark 91, as shown in FIG. 7A. In order to do it automatically, the probe for treatment and diagnosis 3 may also have a mechanism to acquire the positional angle information similar to the three-dimensional model acquiring probe. During the treatment, the results of the treatment effect by the irradiation unit analyzed using the light detector 56 and the controller and analyzer 54 in the main unit 5 are mapped, as shown in FIG. 7B.

The controller and analyzer 54 controls the irradiation of the laser light from the light source 55.

The controller and analyzer 54 calculates the temporal change in the fluorescence intensity in the depth direction based on the calculated temporal change in the distribution of the photosensitizer in the ground state or an optical coefficient in a depth direction, the calculated temporal change in the intensity distribution of light in the depth direction, and the fluorescence intensity on the gum surfaces detected by a light detector 56. First data provided by visualizing the temporal change in the fluorescence intensity is outputted to the display unit 21 and displayed on a right lower area of the display unit 21, as shown in FIG. 19.

The controller and analyzer 54 receives the blood flow volume information from the blood flow volume receiving unit 59, outputs third data for graphing and visualizing the relationship between the blood flow volume and a laser light irradiation time, and displays it at the right upper area on the display unit 21, as shown in FIG. 19. Although a change in the blood flow volume is graphed and displayed here, the blood flow volume may be numerically displayed.

The controller and analyzer 54 receives oxygen saturation information from the oxygen saturation receiving unit 50, outputs fourth data for numerically visualizing the oxygen saturation, and displays it on the display unit 21. Although the image displayed on the display unit 21 includes no oxygen saturation information in FIG. 19, the oxygen saturation information can be displayed by switching a display.

The switch 51 controls on/off of the light irradiation by an operator's operation.

The safeguard 52 detects abnormality or the like of the output of the laser light from the light source 55, and transmits a signal to forcibly stop the output of the laser light to the controller and analyzer 54 once the abnormality or the like is detected.

The blood flow volume receiving unit 59 receives the blood flow volume information measured by the Doppler meter 31 of the probe for treatment and diagnosis 3.

The oxygen saturation receiving unit 50 receives the oxygen saturation information measured by the oxygen saturation meter 32.

### 2. Light-Sensitive Member

As the photosensitizer administered into the oral cavity, a cation formulation that can be bonded to the periodontitis bacteria by an electrostatic interaction and a solution or a gel of a drug taken into the periodontitis bacteria can be used. When the solution of the drug is used, the patient takes it into the oral cavity and then spits it out. When the gel is used, the doctor locally administers it to the patient using a DDS device such as an injection and a microneedle array.

Examples of the cation formulation include methylene blue, toluidine blue, PPA (Phenothiazine), phthalocyanine, C60 and porphyrin.

The drug taken into the periodontitis bacteria includes indocyanine green (ICG).

By administering the photosensitizer into the oral cavity, the photosensitizer is distributed in the gums in the depth direction, and on the surfaces of the teeth, the gums, the plaques and the calculi.

### 3. Flow of Diagnosis and Treatment

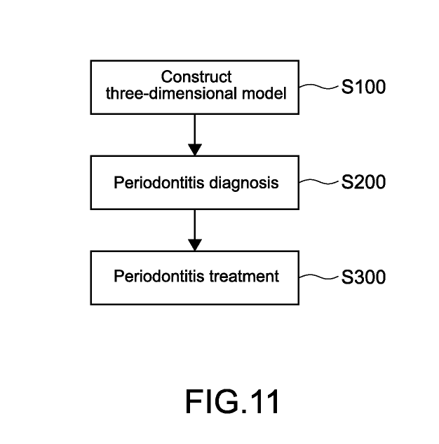

A flow of diagnosis and treatment using the above-described dental apparatus 1 will be described referring to FIG. 11.

As shown in FIG. 11, the three-dimensional model of the oral cavity is firstly acquired (S100). Then, the doctor diagnoses the periodontitis bacteria and explains to the patient (S200). The treatment is done (S300).

Hereinafter, the flow of diagnosis and treatment will be described in detail.

### 3.1. Three-Dimensional Model Acquiring Process

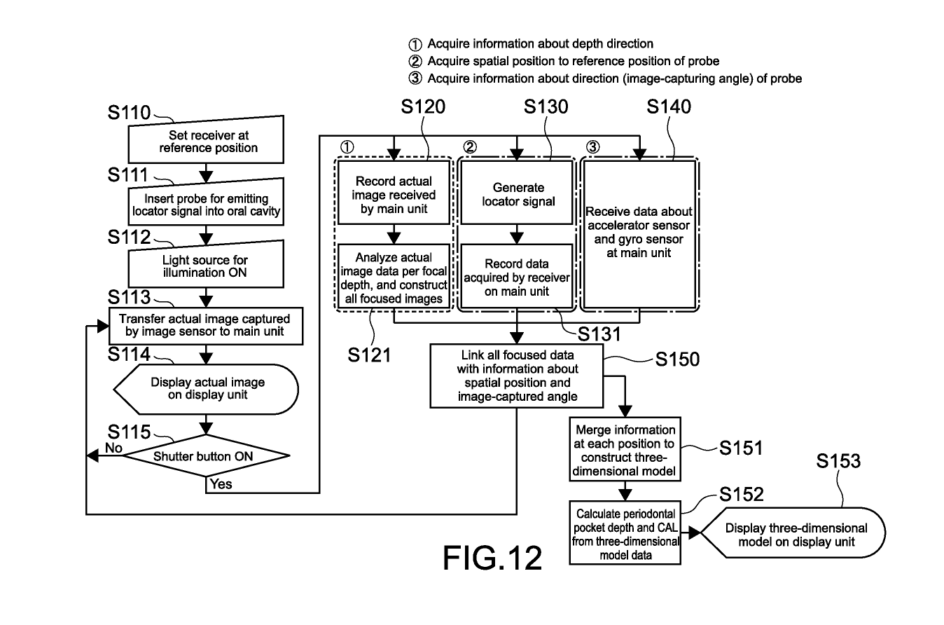

FIG. 12 shows a flow diagram of a processing of acquiring the three-dimensional model. Hereinafter, the processing will be described along the flow shown in FIG. 12.

### (Image Acquisition Preparation Processing and Confirmation Processing, an S110 Number)

Firstly, as shown in FIG. 3, a practitioner inserts the receiver 8 at the left upper central incisor that becomes the reference position for the patient (S110).

Next, the practitioner inserts the three-dimensional model acquiring probe 4 for emitting the locator signal into the patient's mouth (S111), the light source for illumination is turned on, and the illumination unit 41 emits a light (S112).

The CMOS image sensor having an array lens (the image-capturing unit) 42 converts the image data of the image captured site irradiated with the light from the illumination unit 41 to an electrical signal. The image transfer unit 43 transfers this actual image data to the image receiving unit 57 of the main unit 5 (S113).

The controller and analyzer 54 displays the actual image data transmitted from the image transfer unit 43 on the display unit 21 as the actual image (S114).

The controller and analyzer 54 determines whether or not an image-capturing shutter button is depressed by a practitioner (S115).

When the controller and analyzer 54 determines that the image-capturing shutter button is depressed by the practitioner (Yes) at S115, the next step is proceeded.

When the controller and analyzer 54 determines that the image-capturing shutter button is not depressed as the button is not depressed for a predetermined time (No) at S115, the processing is returned to S113, and the similar processing is repeated.

### (Construction Processing of Three-Dimensional Model, an S120 Number)

The image receiving unit 57 of the main unit 5 receives and records the image data transmitted from the image transfer unit 43 of the three-dimensional model acquiring probe 4 (S120). The image processing unit 53 of the main unit 5 analyzes the image data recorded at the image receiving unit 57 per focal depth, and constructs the all focused images (S121).

### (Acquisition Processing of Spatial Positional Data, an S130 Number)

The locator signal generation unit 48 of the three-dimensional model acquiring probe 4 generates a locator signal (S130).

The receiver 8 receives the locator signal, and transmits it as the spatial position information to the positional angle information receiving unit 58 of the main unit 5. The positional angle information receiving unit 58 records the spatial position information (S131). The place where the receiver 8 is placed is set to the reference position. The spatial position information is recorded as the absolute spatial position information from the reference position.

### (Acquisition Processing of Image-Capturing Angle Data by Three-Dimensional Model Acquiring Probe, S140)

The accelerator sensor or the MEMS gyro sensor (angle detector) 44 of the three-dimensional model acquiring probe 4 detects a direction (the image-capturing angle) of the three-dimensional model acquiring probe 4. The image-capturing angle information is transmitted to the positional angle information receiving unit 58 by the angle information transmitter 45 (S140).

(Linking Processing of Image Data with Positional Information, an S150 Number)

The image processing unit 53 links the all focused image data at one time image-captured site with the spatial positional information acquired at S131 and the image-captured angle information acquired at S140 (S150).

Then, the processing is returned to S113, and the similar processing is repeated until the all image data of the oral cavity is acquired.

The image processing unit 53 transmits the all focused image data, the positional information and the image-captured angle information linked to the controller and analyzer 54. The all focused image data, the positional information and the image-captured angle information linked are information about the partial image data of the oral cavity acquired by one-time image-capturing by the CMOS image sensor (the image capturing unit) having an array lens 42 of the three-dimensional model acquiring probe 4.

The controller and analyzer 54 merges the all focused images, the positional information and the positional angle information linked in each of the plurality of the partial image data of the oral cavity to construct the three-dimensional model of the oral cavity (S151).

The controller and analyzer 54 calculates the periodontal pocket depth and a Clinical Attachment Level (CAL) from the image data of the three-dimensional model (S152).

The controller and analyzer 54 displays the three-dimensional model constructed on the display unit 21 (S153).

Also, the controller and analyzer 54 displays the periodontal pocket depth and the CAL calculated by the controller and analyzer 54 on the display unit 21.

Concerning the acquisition of the image data of the left upper central incisor at which the receiver 8 is disposed, the receiver 8 may be disposed at a right upper central incisor and the reference position may be set thereto to acquire the image data.

### 3.2. Diagnosis of Periodontitis

The practitioner diagnoses the status of the oral cavity and periodontitis severity based on the three-dimensional model of the oral cavity, the periodontal pocket depth and the CAL.

In the related art, the periodontal pocket depth and the CAL are determined by using a proving method. However, in the proving method, an instrument having a memory on a tip, which is called as a probe, is inserted between the tooth and the gum, whereby the gum is damaged by the probe, which may often cause the bleed. Upon the bleeding, bacteria may enter into the blood.

In contrast, according to the first embodiment, the periodontal pocket depth and the CAL are determined by the image data without using the probe, and no bleeding is involved.

The practitioner show the three-dimensional model of the oral cavity displayed on the display unit 21 to the patient, and explains the status of the oral cavity and treatment policy.

### 3.3. Treatment of Periodontitis

Next, the treatment of periodontitis will be described. The periodontitis is treated using the probe for treatment and diagnosis 3 while the receiver 8 used in the acquisition of the three-dimensional model is disposed at the upper central incisor.

Upon the treatment of the periodontitis, the receiver is used as the reference position similar to upon the acquisition of the three-dimensional model, and the description is therefore omitted. Also, the locator signal generator, the acceleration sensor or the MEMS gyro sensor 44 is disposed at the probe for treatment and diagnosis 3 similar to three-dimensional model acquiring probe 4. From these, the positional information of the treatment site is provided.

Hereinafter, a method of acquiring the image for visualizing the temporal change in the disinfection status of the periodontitis bacteria of the gum in the depth direction will be described.

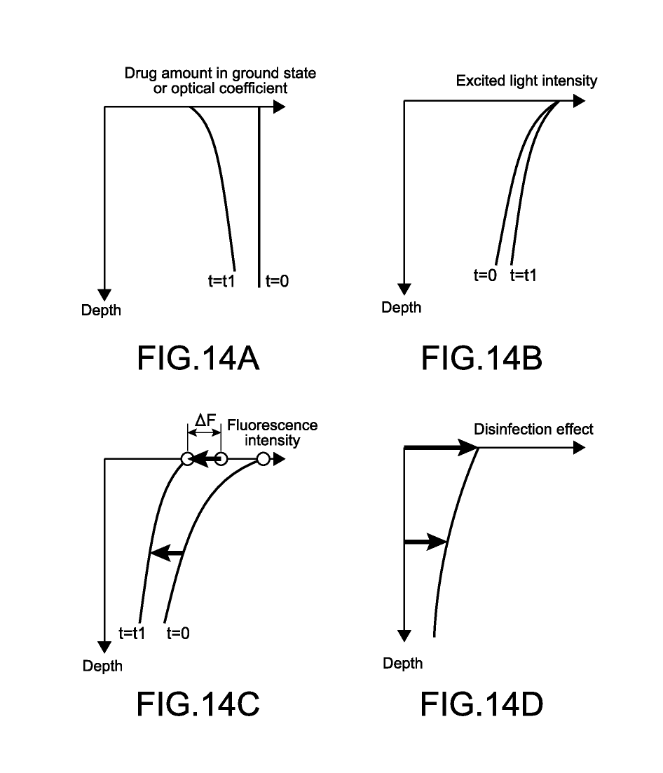

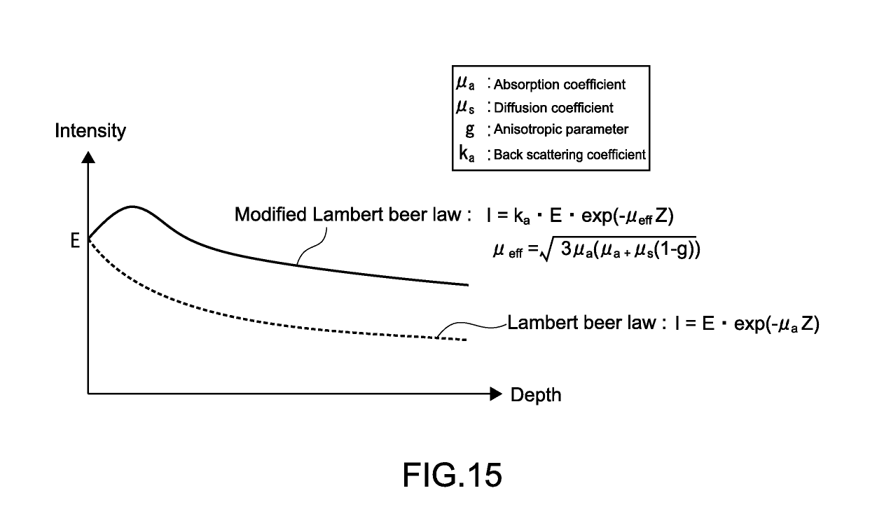

FIG. 13 is a flow diagram showing a processing of acquiring an image showing a disinfection effect of the gum in a depth direction upon the treatment. FIGS. 14A to 14D are each a graph for illustrating a method of estimating the disinfection effect. Hereinafter, the method will be described in accordance with the flow shown in FIG. 13 using FIGS. 14A to 14D as appropriate.

### (Image Acquisition Preparation and Fluorescence Acquisition Processing, an S310 Number)

Firstly, the photosensitizer is locally administered at the diseased site (S310).

The probe for treatment and diagnosis 3 is fixed while the probe for treatment and diagnosis 3 is in contact with the surfaces of the gums (S311).

Thereafter, when the switch for controlling the irradiation of the light from the light irradiation probe for treatment and diagnosis 34 is turned on by the practitioner (S312), the treatment and diagnosis light that is the excited light is emitted from the light irradiation probe for treatment and diagnosis 34. The treatment and diagnosis light irradiates the diseased site, and diffuses and reflects on the surfaces of the gums, the plaques or the calculi.

Two light-receiving probes 33, 33 of the probe for treatment and diagnosis 3 receive diffused and reflected light on the surfaces of the gums, the plaques, and the calculi by irradiating the treatment and the diagnosis light, and fluorescence emitted from the surfaces of the gums, the plaques, and the calculi (S320), and lead the diffused and reflected light and the fluorescence of the excited light to the light detector 56 of the main unit 5.

The light detector 56 splits the diffused and reflected light of the excited light and the fluorescence, detects the diffused and reflected light intensity on the surfaces of the gums and the plaques from the diffused and reflected light of the excited light, and also detects the fluorescence intensity of the surfaces of the gums, the plaques or the calculi from the fluorescence (S321).

The light detector 56 records the diffused and reflected light intensity and the fluorescence intensity of the excited light (S322). The light detector 56 transmits the diffused and reflected light intensity information and the fluorescence intensity information of the excited light to the controller and analyzer 54.

(Calculation Processing of Fluorescence Distribution of Gums in Depth Direction at Time t1=0, an S330 Number)

The controller and analyzer 54 determines that the time is t1=0 or not (S330).

At S330, when the time is t1=0 (Yes), the controller and analyzer 54 calculates the optical coefficient of gum tissues and the plaques from the intensity of the former excited light that is diffused and reflected on the surfaces of the gums and the intensity of the diffused and reflected light of the excited light including an absorption effect by the photosensitizer (S331).

Next, the controller and analyzer 54 estimates and calculates the intensity of the excited light at the gums in the depth direction from the optical coefficient calculated at S331 (S332). By the calculation, a relationship between the gums in the depth direction and the intensity of the excited light when t=0, as shown in a graph of FIG. 14B. In FIG. 14B, the deeper the depth is, the more the excited light difficult to be arrived is; thus, the deeper the depth is, the smaller the intensity of the excited light is. When t=0, the distribution of an optical constant or an amount of a drug is regarded as constant in a uniform tissue model. Its value can be measured in advance in separate experiment.

Next, the controller and analyzer 54 estimates and records the fluorescence intensity in each depth from the distribution of the excited light intensity in the depth direction acquired at S332 and the distribution of the amount of the photosensitizer in a ground state in a depth direction calculated in advance or the distribution of the optical coefficient (S333). By the calculation, a relationship between the gums in the depth direction and the fluorescence intensity when t=0, as shown in a graph of FIG. 14C.

(Calculation Processing of Fluorescence Distribution of Gums in Depth Direction when Time t1≠0, an S340 Number)

At S330, when the time is t1 not equals to 0 (No), the controller and analyzer 54 calculates a difference ΔF (a bleaching amount on the surfaces of the gums) between the fluorescence intensity on the surfaces of the gums at the time t1 and the fluorescence intensity on the surfaces of the gums at the time (t1−Δt) detected by the light-receiving probe 33 (S340).

Next, the controller and analyzer 54 estimates the distribution of the amount of the drug bleached amount during Δt in the depth direction based on the difference ΔF of the fluorescence intensity on the surfaces of the gums calculated at S340 and the distribution of the excited light intensity at the time (t1−Δt) (S341).

Next, the distribution of the fluorescence intensity in the depth direction at time t1 is estimated and calculated (S342). By the calculation, the relationship between the gums in the depth direction and the fluorescence intensity at t=t1 is provided as shown in FIG. 14C.

Next, the controller and analyzer 54 estimates and calculates the distribution of the optical coefficient in the depth direction taking the absorbed drug at the time t1 into consideration (S343). By the calculation, the relationship between the gums in the depth direction and the optical coefficient at t=t1 is provided as shown in FIG. 14A.

Next, the controller and analyzer 54 estimates, calculates, and record the distribution of the excited light intensity in the depth direction at the time t1 (S344). By the calculation, the relationship between the gums in the depth direction and the laser light intensity at t=t1 is provided as shown in FIG. 14B.

(Signal Generation Processing Showing Disinfection Status in Gums in Depth Direction after t1 is Elapsed from Start of Treatment, an S350 Number)

After S342, parallel to S343 to S344 processing, the controller and analyzer 54 calculates a difference between the fluorescence intensities at the time 0 and the time t1 in each depth position (S350).

Next, the controller and analyzer 54 plots the calculation result at S350 in the depth direction, and outputs first data for visualizing the disinfection effect of the periodontitis bacteria after t1 is elapsed as shown in FIG. 14D (S351). The first data is for visualizing the elapsed change in the fluorescence intensity based on the fluorescence detected by the light detector.

The display unit 21 displays the image of the first data. The image 214 displayed at the right lower area of the display unit 21 of the monitor 2 shown in FIG. 19 is the first data.

Nest, at S360, the controller and analyzer 54 sets t1=t1+Δt.

Next, the controller and analyzer 54 determines that the light irradiation by the practitioner is turned off or not (S361).

At S361, it is determined as off (Yes), the treatment is ended (S362).

At S361, it is determined as not off (No), the flow is returned to S320, and the processing is repeated.

By the above-described processing, the image 214 is displayed on the right lower area of the display unit 21 of the monitor 2 as shown in FIG. 19. In the image 214, the temporal change in the disinfection status of the periodontitis bacteria of the gums in the depth direction is visualized. The practitioner and the patient can confirm the disinfection status of the periodontitis bacteria in real time by the image 214.

The practitioner observes the image and treats to disinfect the periodontitis bacteria with certainty. Therefore, there is no chance to be insufficient disinfection due to the shortage of the laser light irradiation time. Thus, it does not take a long time to cure the periodontitis completely by the sufficient laser light irradiation, and the treatment period can be shortened.

The patient can confirm the disinfection status of the periodontitis bacteria in real time, can realize the disinfection effect, and will positively treat the periodontitis bacteria.

Also in the first embodiment, the receiver that generates the locator signal upon the treatment to acquire the positional information about the treatment site, and the positional information is correlated with the positional information provided when the three-dimensional model is acquired. In this way, the results of the treatment effect during the irradiation are mapped gradationally as shown in FIG. 7B, and it is possible to treat the treatment site while the treatment effect is confirmed in real time. The treatment site and the treatment result can be recorded and controlled.

### Alternative Embodiment

As the above-mentioned probe for treatment and diagnosis 3 uses as the treatment and diagnosis light not only laser light, but also a light-emitting diode light and a light provided by cutting a light from a lamp light source with an optical filter.

In addition, the above-mentioned probe for treatment and diagnosis 3 guides a light using fibers having high transmittance and good flexibility such as quartz, POF (Plastic Optical Fiber) and the like.

In the above-described embodiment, the positional information of the treatment site is provided by the receiver upon the treatment, and the treatment site is defined therefrom as the three-dimensional model. Alternatively, the practitioner may plot and record the treatment site on the three-dimensional model of the oral cavity displayed on the display unit as shown in FIG. 7A.

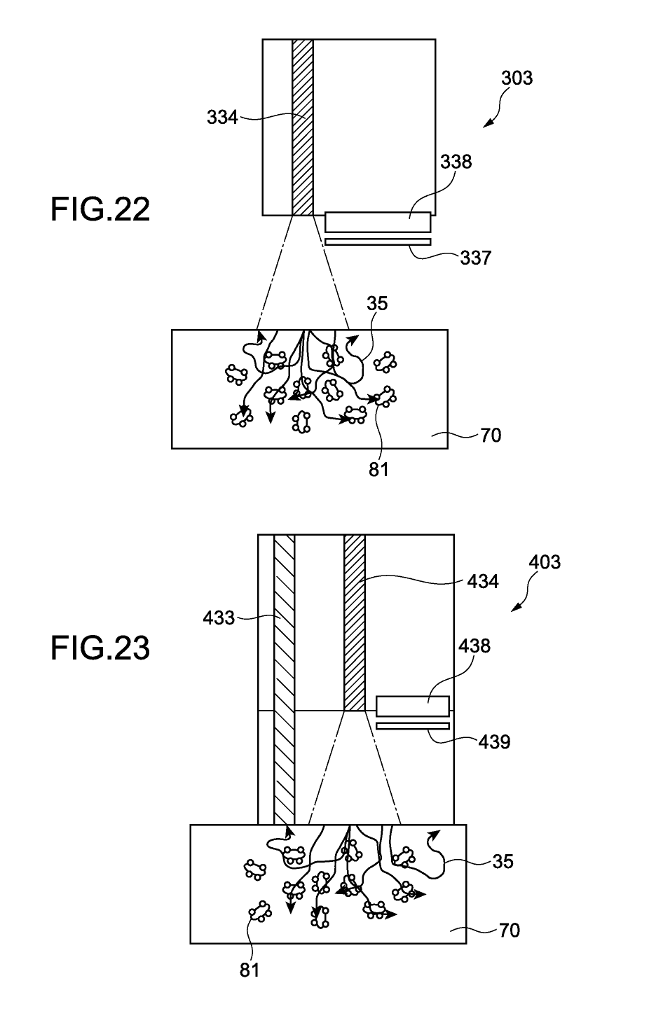

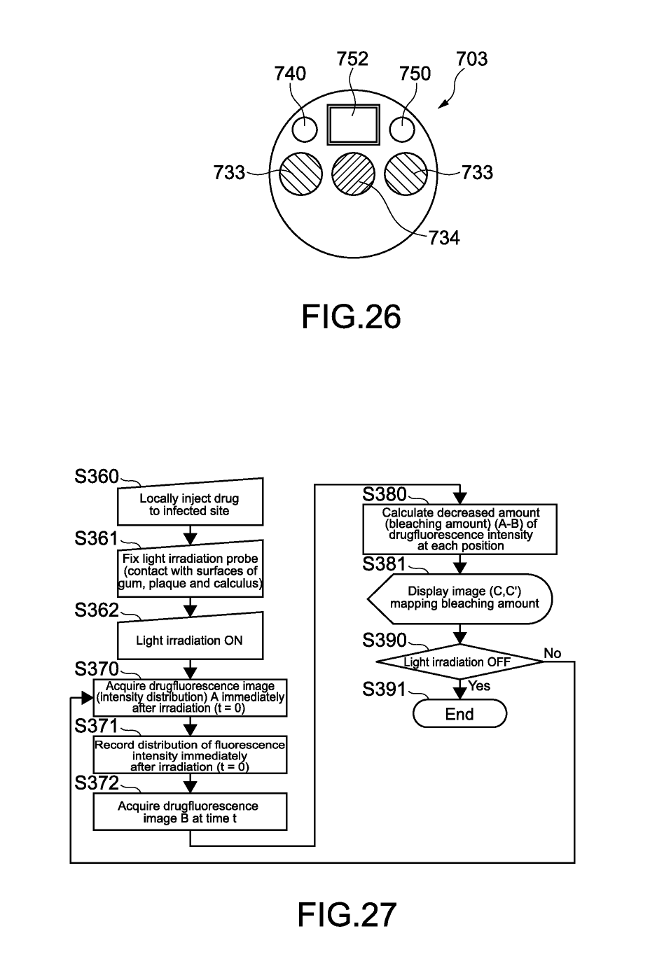

In addition to the above-described probe for treatment and diagnosis 3, probes for treatment and diagnosis shown in FIGS. 22 to 26 can be used. FIGS. 22 to 25 each shows a schematic sectional diagram of an alternative probe for treatment and diagnosis. FIG. 26 shows a tip diagram of the alternative probe for treatment and diagnosis.

The configurations similar to the above-described embodiments are denoted by the same reference numerals, and thus detailed description thereof will be hereinafter omitted.

As shown in FIG. 22, an alternative probe for treatment and diagnosis 303 is a non-contact type, and includes a light irradiation probe for treatment and diagnosis 334, a CMOS image sensor, a CCD image sensor or imaging fibers 338, and an optical filter 337.

The probe for treatment and diagnosis 303 differs largely from the probe for treatment and diagnosis 3 according to the first embodiment in that the image sensor 338 and the optical filter 337 are disposed instead of the light-receiving probe 33 of the probe for treatment and diagnosis 3 according to the first embodiment. The light irradiation probe for treatment and diagnosis 334 emits the treatment and diagnosis light similar to the light irradiation probe for treatment and diagnosis 34 according to the first embodiment.

The optical filter 337 transmits only fluorescence. Specifically, the optical filter 337 transmits only fluorescence contained in the light from the gum 70, the plaques or the calculi and emitted to the excited light. The transmitted fluorescence is incident on the image sensor 338. In the image sensor 338, the fluorescence intensity is mapped gradationally. When the imaging fibers 338 are used, the optical filter 337 may be disposed at a later part.

A light emitted end face of the light irradiation probe for treatment and diagnosis 334 is disposed apart from the gum 70 at a predetermined distance. In this way, the light can be irradiated on a larger area than a core diameter than the light irradiation probe for treatment and diagnosis 334. In contrast, in the probe for treatment and diagnosis 3 according to the first embodiment, a light emitted end face of the light irradiation probe for treatment and diagnosis 34 is in contact with the gum 70, so that the light is irradiated on an area similar to a core diameter of the light irradiation probe for treatment and diagnosis 34.

As shown in FIG. 23, a probe for treatment and diagnosis 403 as an another embodiment is a contact type, and includes a light irradiation probe for treatment and diagnosis 434, a light receiving probe 433, an image sensor 438, and a diffusion plate 439.

In the probe for treatment and diagnosis 403, fluorescence from the gum 70, the plaques or the calculi emitted to the excited light is received at the image sensor or the imaging fibers 438 and the diffusion plate 439 as well as the light-receiving probe 433. The light irradiation probe for treatment and diagnosis 434 emits a light for treatment and diagnosis similar to the light irradiation probe for treatment and diagnosis 34 according to the first embodiment. The light-receiving probe 433 is similar to the light-receiving probe 33 according to the first embodiment.

The diffusion plate 439 diffuses the light emitted at the gum 70 side, and broads the light incident on the image sensor 438. In the image sensor 438, the fluorescence intensity is mapped gradationally.

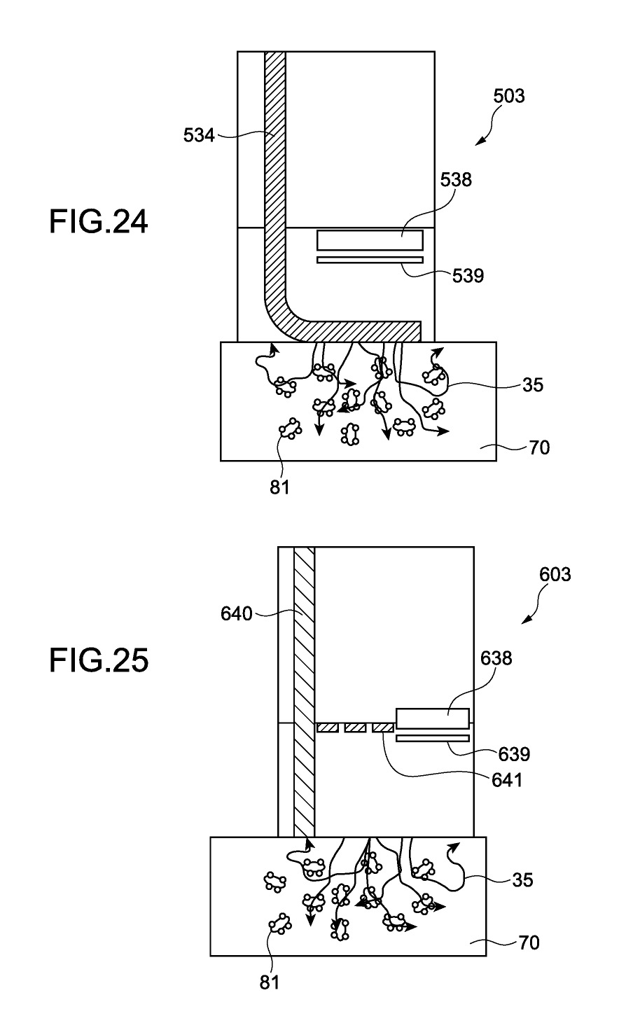

As shown in FIG. 24, a probe for treatment and diagnosis 503 as a still another embodiment is a contact type, and includes a light irradiation probe for treatment and diagnosis 534, an image sensor or an imaging fiber 538, and an optical filter 539. The optical filter 539 is similar to the image sensor 338 according to the above-described alternative embodiment.

In the probe for treatment and diagnosis 503, the light irradiation probe for treatment and diagnosis 534 is a lateral irradiation or all direction irradiation probe, and has a curved tip. In this way, a wide area of the gum 70 can be irradiated with the treatment and diagnosis light.

As shown in FIG. 25, a probe for treatment and diagnosis 603 as a still further another embodiment is a contact type, and includes a light-emitting diode 641, a light-receiving probe 640, an image sensor or imaging fibers 638, and an optical filter 639. The light-receiving probe 640 is similar to the light-receiving probe 33 according to the first embodiment. The optical filter 639 is similar to the optical filter 337 according to the above-described alternative embodiment. The image sensor 638 is similar to the image sensor 338 according to the above-described alternative embodiment.

In the probe for treatment and diagnosis 603, a light-emitting diode light is used instead of the laser light, and a light-emitting diode 641 is disposed as a light source.

As shown in FIG. 26, a probe for treatment and diagnosis 703 as a still further another embodiment includes two light-receiving probes 733, a light irradiation probe for treatment and diagnosis 734, an air outlet 750, a CMOS image sensor having an array lens or imaging fibers 752, and an illumination unit 740. The light-receiving probe 733 is similar to the light-receiving probe 33 according to the first embodiment.

The light irradiation probe for treatment and diagnosis 734 guides and emits a polychromatic light provided by collecting the treatment and diagnosis light, a light of the Doppler meter and a light of the oxygen saturation meter.

The air outlet 750 that is an air blowing unit blows air to the diseased site. Since main bacteria engaged in the periodontitis is anaerobes bacteria (strictly anaerobic bacteria or facultative anaerobic bacteria), the diseased site is irradiated with the treatment and diagnosis light while blowing air during the treatment, thereby further improving the disinfection effect.

The CMOS image sensor having an array lens or imaging fibers 752 can acquire all focused images similar to the CMOS image sensor having an array lens 42 of the three-dimensional model acquiring probe 4 according to the first embodiment. Thus, the probe may have both of a three-dimensional model acquiring function and a treatment function.

The illumination unit 740 emits a light for illuminating the image captured site upon the image-capturing.

In the above-described embodiments, the light emitted from the probe for treatment and diagnosis 3 is a monochromatic light including a treatment and diagnosis laser light alone, but may be a polychromatic light including the treatment and diagnosis laser light and other light.

When the light emitted from the light irradiation probe for treatment and diagnosis 34 is the polychromatic light, a wavelength can be changed depending on a depth of a periodontitis infected site or the site can be irradiated with a light having different wavelengths. For example, methylene blue absorbs an excited light having a wavelength of 670 nm, and has a good balance in terms of absorption efficiency of a drug and reachability to a tissue. However, an infected site at a certain depth, for example, at a depth of 2 mm or more, may not be sufficiently treated by methylene blue. Then, when the polychromatic light having wavelengths of 670 nm and 830 nm is used as the light emitted from the light irradiation probe for treatment and diagnosis, the light can reach deep into the diseased site, thereby treating a deep part, although the light having a wavelength of 830 nm decreases the light absorption efficiency of the drug.

FIG. 38 is a graph showing a relationship between the absorption coefficient of the photosensitizer and the light reachability of the polychromatic light. An abscissa axis represents the wavelength, and a longitudinal axis represents the absorption coefficient or the light reachability. In FIG. 38, a solid line represents the absorption coefficient of the photosensitizer, and a dotted line represents the light reachability of the polychromatic light.

As shown in FIG. 38, by using the polychromatic light, e.g., the light having not good light absorption efficiency but being capable of reaching the deep part such as 3 mm, the PDT can be implemented to a deeper area where a short wavelength cannot reach.

In the above-described embodiment, the probe for treatment and diagnosis 3 guides the light used in the Doppler meter 31 and the light of the oxygen saturation meter 32 as well as the laser light for treatment and diagnosis. In the above-described embodiments, the light irradiation probe for treatment and diagnosis 34 uses, including, but not limited to, the monochromatic light as only the treatment and diagnosis light. For example, the light irradiation probe for treatment and diagnosis may be bundle fibers that bundle three cores of optical fibers for treatment and diagnosis, optical fibers for a Doppler meter, and optical fibers for oxygen saturation meter. Each fiber may collect each light to irradiate the diseased site with the polychromatic light from the light irradiation probe for treatment and diagnosis.

Also, the treatment and diagnosis light, the light of the Doppler meter and the light of the oxygen saturation meter may be collected at one fiber.

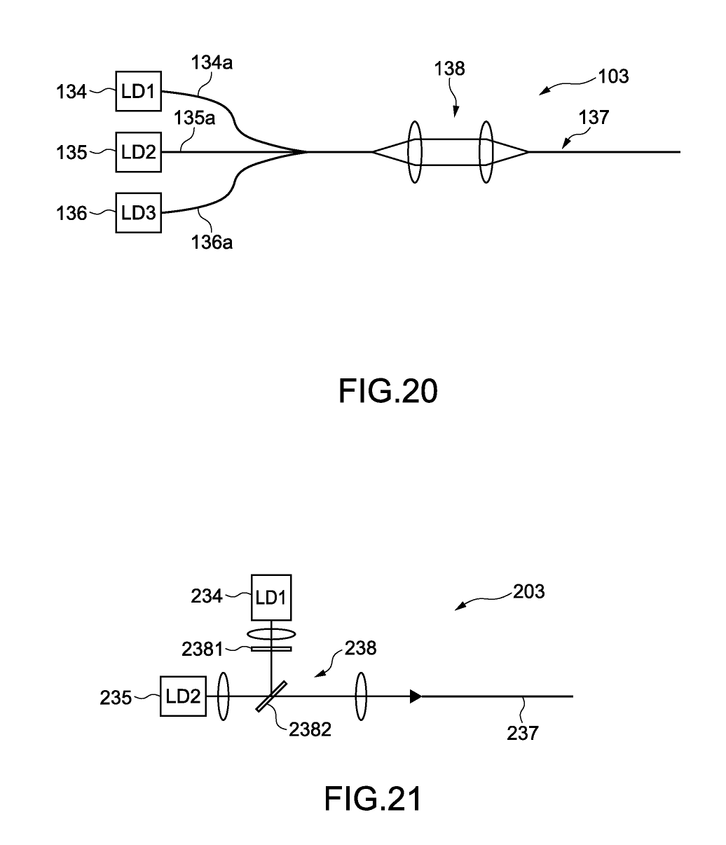

As shown in FIG. 20, the light emitted from the three core bundle of fibers 134a that leads the treatment and diagnosis light emitted from the a laser diode light source 134 of the treatment and diagnosis light, a fiber 135a that leads the light of the Doppler meter emitted from a laser diode light source 135 of the light of the Doppler meter, and fibers 136a that lead the light of the oxygen saturation meter emitted from a laser diode source 136 of the light of the oxygen saturation meter may be collected into single core fibers 138. In this case, the entirely same diseased site will be irradiated with a three-colored light.

As shown in FIG. 21, the treatment and diagnosis light emitted from a light source 234 for the treatment and diagnosis light and the light of the Doppler meter emitted from a light source 235 for the light of the Doppler meter may be collected into single core fibers 237 through an optical system 238 having a magnification providing a desired NA and a core diameter.

Although the three-dimensional model of the oral cavity is acquired using the three-dimensional model acquiring probe in the above-described embodiments, but not limited to, x-rays may be used to acquire the image of the oral cavity.

Although there is shown the image where the temporal change in the disinfection status of periodontitis bacteria on the gums in the depth direction is visualized in the above-described embodiments, there may be shown the image where the temporal change in the disinfection status of periodontitis bacteria distributed in the depth direction of the plaques or the calculi attached to the teeth and the gums is visualized.

Next, a second embodiment will be described.

### Second Embodiment

Although there is shown the image where the temporal change in the disinfection status of periodontitis bacteria distributed in the depth direction of the gums is visualized in the first embodiment, there may be shown the image where the temporal change in the disinfection status of periodontitis bacteria on the surfaces of the gums and the teeth. Hereinafter, it will be described as the second embodiment.

The dental apparatus according to the second embodiment is the same as that according to the first embodiment. Hereinafter, the method of acquiring the image where the temporal change in periodontitis bacteria on the surfaces of the gums and the teeth is visualized will be described.

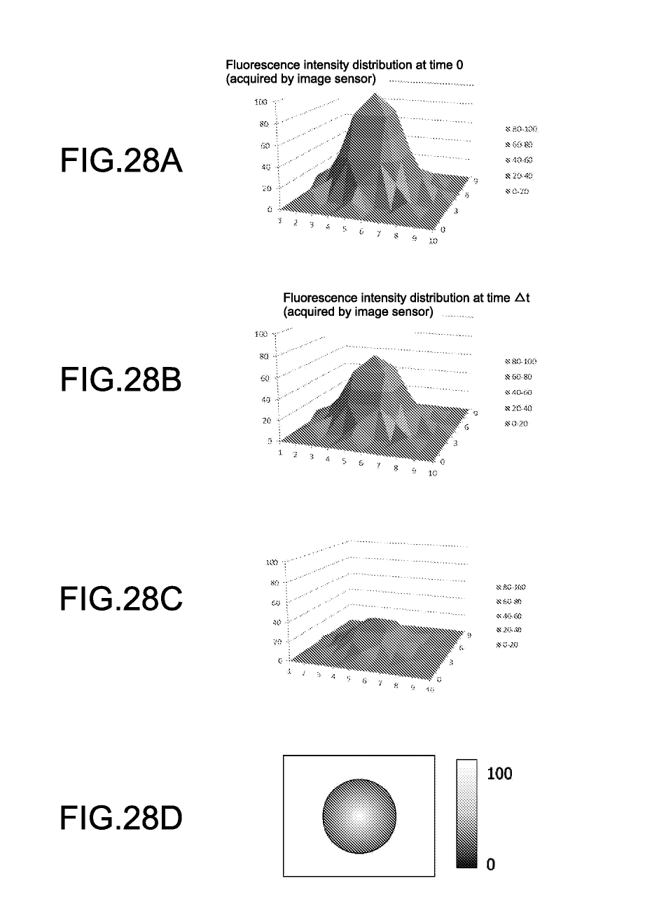

FIG. 27 is a flow diagram showing a flow of a processing of acquiring the image showing the disinfection effect of periodontitis bacteria on the surfaces of the gums and the teeth upon the treatment. FIGS. 28A to 28C are each an image diagram for constructing a mapping of the disinfection effect. FIG. 28D is a mapping of the disinfection effect. Hereinafter, referring to the flow shown in FIG. 27 and, as appropriate, FIGS. 28A to 28D, visualizing the disinfection status will be described.

### Image Acquisition Preparation, an S360 Number

Firstly, the photosensitizer is locally administered at the diseased site (S360).

The probe for treatment and diagnosis 3 is fixed while the probe for treatment and diagnosis 3 is in contact with the surfaces of the gums (S361). Herein, the probe for treatment and diagnosis 3 is contacted with the surface of the gums as an example, but may be contacted with the surfaces of the teeth.

Thereafter, when the switch for controlling the irradiation of the light from the light irradiation probe for treatment and diagnosis 34 is turned on by the practitioner (S362), the treatment and diagnosis light that is the excited light is emitted from the light irradiation probe for treatment and diagnosis 34. The laser light irradiates the diseased site.

### Fluorescence Acquisition Processing, an S370 Number

Two light-receiving probes 33, 33 of the probe for treatment and diagnosis 3 receive fluorescence from the surfaces of the gums emitted to the excited light and lead the fluorescence to the light detector 56 of the main unit 5. The light detector 56 detects the fluorescence led. Based on the fluorescence detected by the light detector 56, the controller and analyzer 54 acquires a drugfluorescence image (fluorescence intensity distribution) immediately after the irradiation (t=0) as shown in FIG. 28A (S370).

The controller and analyzer 54 records the fluorescence intensity distribution on the gum surfaces immediately after the irradiation (t=0) as shown in FIG. 28B (S371).

The light detector 56 of the main unit 5 acquires the drugfluorescence image (fluorescence intensity distribution) at the time t=t1 as shown in FIG. 28B (S372). The light detector 56 transmits fluorescence intensity information at the time t=t1 to the controller and analyzer 54.

### Calculation Processing of Fluorescence Intensity, an S380 Number

The controller and analyzer 54 calculates a decreased amount (a bleaching amount) of the drugfluorescence intensity from the fluorescence intensity information at the time t=t1 (S380).

The controller and analyzer 54 outputs first data for visualizing the bleaching amount during the time t1 as shown in FIGS. 28C and 28D, and displays it on the display unit 21 (S381).

Here, FIG. 28C is the image for sterically visualizing the bleaching amount on the surface of the gums. FIG. 28D is the image for planar visualizing the bleaching amount on the surface of the gums. The lighter (whiter) the shade is, the higher the bleaching amount is. The darker the shade is, the lower the bleaching amount is.

### End Processing, an S390 Number

Next, it determines that the light irradiation by the practitioner is turned off or not (S390).

At S390, it is determined as off (Yes), the treatment is ended.

At S390, it is determined as not off (No), the flow is returned to S370, and the processing is repeated.

As described above, according to the second embodiment, there is provided the image for visualizing the temporal change in the disinfection status of periodontitis bacteria that are present on teeth or gum surfaces, i.e., are two-dimensionally distributed, the practitioner and the patient can confirm the disinfection status of the periodontitis bacteria in real time.

Both of the disinfection status of the gums in the depth direction according to the first embodiment and the disinfection status of the gum surfaces according to the second embodiment may be displayed on the display unit 21.

### Third Embodiment

In the above-described embodiment, the treatment is done after the three-dimensional model of the oral cavity is acquired. Alternatively, the treatment may be done without acquiring the three-dimensional model of the oral cavity. For example, the three-dimensional model of the oral cavity is not displayed on the monitor upon the treatment, and the actual image of the sites being treated and the disinfection status of the sites irradiated with the treatment and diagnosis light may be displayed.

Hereinafter, the disinfection status of the gum surfaces is displayed as an example. Alternatively, the image showing the disinfection status in the depth direction may be displayed using the calculation processing according to the first embodiment.

### Configuration of Dental Apparatus

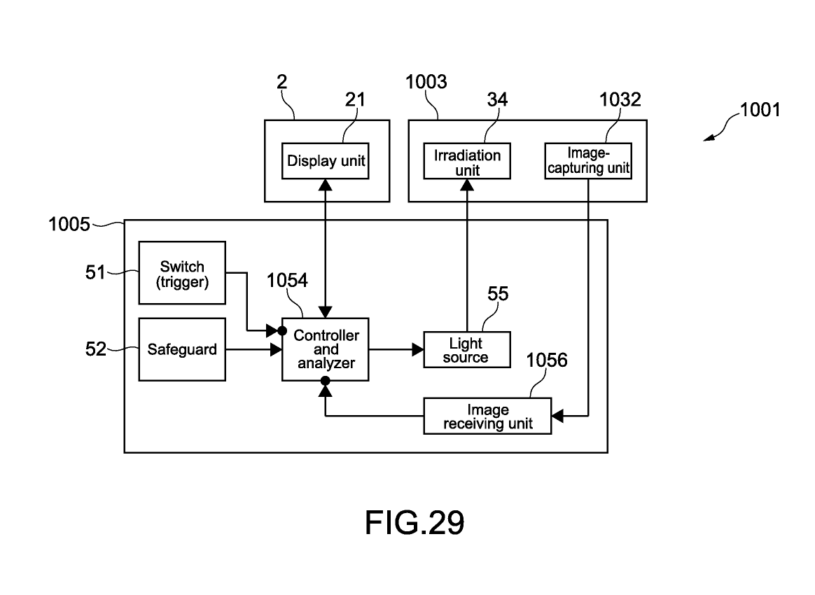

FIG. 29 shows a functional block diagram of the dental apparatus according to a third embodiment. The configurations similar to the above-described embodiments are denoted by the same reference numerals, and thus detailed description thereof will be hereinafter omitted. Mainly, only different points will be described.

As shown in FIG. 29, a dental apparatus 1001 includes the monitor 2, a main unit 1005, and a probe for treatment and diagnosis 1003.

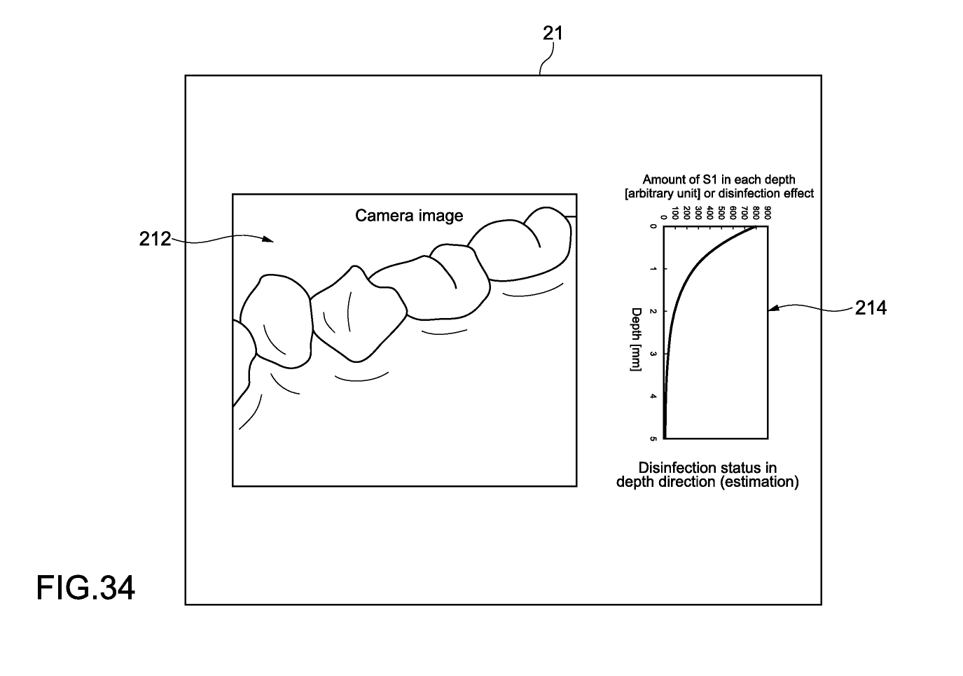

FIG. 34 shows an example of an image displayed on the display unit 21 of the monitor 2.

At a left area in the display unit 21, the actual image (the camera image) 212 of the sites being treated using the probe for treatment and diagnosis 1003 is displayed.

At a right area on the display unit 21, a graph image 214 is displayed. The graph image 214 shows the temporal change in the disinfection status of periodontitis bacteria of the gums in a depth direction. In the image 214, S1 denotes the photosensitizer 94 in the ground state. An ordinate axis of the image 214 may be an indicator of a disinfection effect calculated in accordance with a flow diagram shown in FIG. 13.

The probe for treatment and diagnosis 1003 includes an irradiation unit 34 and an image-capturing unit 1032.

The image-capturing unit 1032 converts fluorescence image data emitted from the oral cavity to an electrical signal, and transmits the resultant image data to an image receiving unit 1056 of the main unit 1005.

The main unit 1005 includes the light source 55, the image receiving unit 1056, a controller and analyzer 1054, the switch 51 and the safeguard 52.

The image receiving unit 1056 receives the actual image data of the treatment site in the oral cavity at the moment the data is transmitted from the image-capturing unit 1032 of the probe for treatment and diagnosis 1003.

The controller and analyzer 1054 gradationally maps the fluorescence intensity from the actual image data acquired at the image-capturing unit 1032, outputs first data for visualizing the temporal change in the fluorescence intensity shown in FIG. 28D to the display unit 21, and displays the image provided by visualizing the temporal change in the fluorescence intensity on the display unit 21.

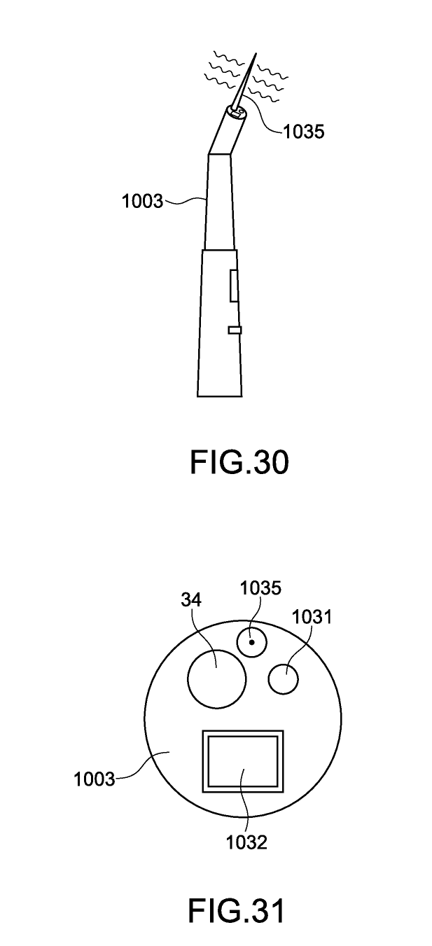

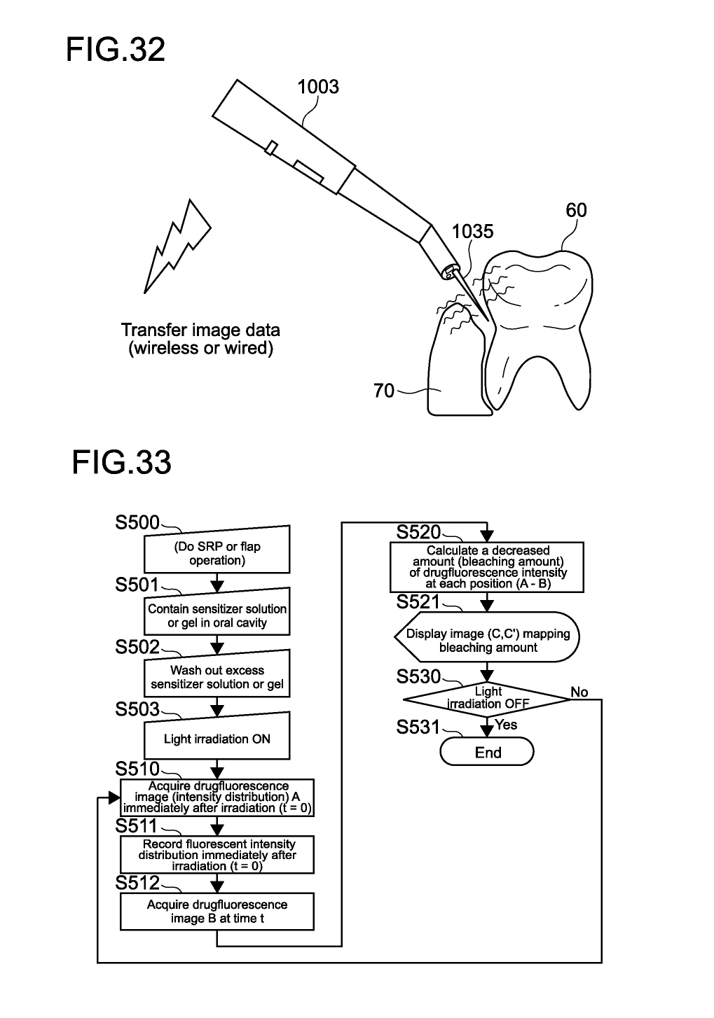

FIG. 30 is an overall view of the probe for treatment and diagnosis 1003. FIG. 32 shows the treatment using the probe for treatment and diagnosis 1003.

As shown in FIG. 30, the probe for treatment and diagnosis 1003 is a stick having a gripper. The probe for treatment and diagnosis 1003 includes a needle-shaped light irradiation unit 1035 for treatment and diagnosis at a tip.

The probe for treatment and diagnosis 1003 is a lateral irradiation type where the treatment and diagnosis light is emitted from a lateral of the needle-shaped light irradiation unit 1035.

As shown in FIG. 32, the needle-shaped light irradiation unit 1035 is inserted into the periodontal pocket formed between the gum 70 and the tooth 60.

FIG. 31 shows a base surface of the needle-shaped light irradiation unit 1035.

As shown in FIG. 31, on the base surface of the needle-shaped light irradiation unit 1035, the light irradiation probe for treatment and diagnosis 34, a CMOS or CCD image sensor having a BPF (Band-Pass Filter) as the image-capturing unit, and an air outlet 1031.

The BPF passes the frequencies within the necessary range, and does not pass the rest of the frequencies. Herein, the BPF passes only the fluorescence emitted from the oral cavity when the photosensitizer is irradiated with the excited light.

The air outlet 1031 is for blowing air to the diseased site. Since main bacteria engaged in the periodontitis is anaerobes bacteria (strictly anaerobic bacteria or facultative anaerobic bacteria), the diseased site is irradiated with the treatment and diagnosis light while blowing air during the treatment, thereby further improving the disinfection effect.

The CMOS or the CCD image sensor 1032 having the BPF passes the fluorescence of the incident light, and image-captures the fluorescence.

### Image Acquisition Processing

Next, the method of acquiring the image where the temporal change in periodontitis bacteria distributed on the surfaces of the gums and the teeth is visualized using the dental apparatus 1001 according to the third embodiment will be described.

FIG. 33 is a flow diagram showing a flow of a processing of acquiring the image showing the disinfection effect of periodontitis bacteria distributed on the surfaces of the gums and the teeth upon the treatment according to the third embodiment. FIGS. 28A to 28C are each an image diagram for constructing a mapping of the disinfection effect. FIG. 28D is a mapping of the disinfection effect. Hereinafter, referring to the flow shown in FIG. 33 and, as appropriate, FIGS. 28A to 28D, visualizing the disinfection status will be described.

In the third embodiment, after scaling (SRP, calculi removal) or a flap operation for incising the gum is done to expose the sites infected by the periodontitis bacteria, the periodontitis bacteria are disinfected by laser light.

### Image Acquisition Preparation, an S500 Number

The scaling or the flap operation is done on the diseased site (S500).

Next, after a solution or a gel of the photosensitizer is administered into the oral cavity (S501), an excess solution or gel of the photosensitizer is washed out (S502).

The probe for treatment and diagnosis 1003 is disposed around the diseased site, and the practitioner turns on the switch for controlling the irradiation of the light from the light irradiation probe for treatment and diagnosis 34 (S503), the excited light is emitted from the light irradiation probe for treatment and diagnosis 34. The laser light irradiates the diseased site.

### Fluorescence Acquisition Processing, an S510 Number

The CMOS or the CCD image sensor 1032 having the BPF of the probe for treatment and diagnosis 1003 acquires fluorescence data from the gum surfaces emitted to the excited light, and transmits the fluorescence data to the image receiving unit 1056 of the main unit 1005.

The image receiving unit 1056 acquires a drugfluorescence image (fluorescence intensity distribution) immediately after the irradiation (t=0) as shown in FIG. 28A (S510). The image receiving unit 1056 transmits fluorescence intensity information at the time t=t0 to the controller and analyzer 1054.

The controller and analyzer 1054 records a gum surface distribution of the fluorescence intensity immediately after the irradiation (t=0) (S511).

The image receiving unit 1056 acquires a drugfluorescence image (fluorescence intensity distribution) at the time t=t1 as shown in FIG. 28B (S512). The image receiving unit 1056 transmits fluorescence intensity information at the time t=t1 to the controller and analyzer 1054.

### Calculation Processing of Fluorescence Intensity, an S520 Number

The image receiving unit 1056 calculates a decreased amount (a bleaching amount) of the drugfluorescence intensity from the fluorescence intensity information at t=0 and the fluorescence intensity information at the time t=t1 (S520).

The controller and analyzer 1054 outputs first data for visualizing the bleaching amount during the time t1 as shown in FIGS. 28C and 28D, and displays it on the display unit 21 (S521).

Here, FIG. 28C is the image for sterically visualizing the bleaching amount on the surface of the gums. FIG. 28D is the image for planar visualizing the bleaching amount on the surface of the gums. The lighter (whiter) the shade is, the higher the bleaching amount is. The darker the shade is, the lower the bleaching amount is.

### End Processing, an S530 Number

Next, it determines that the light irradiation by the practitioner is turned off or not (S530).

At S530, it is determined as off (Yes), the treatment is ended.

At S530, it is determined as not off (No), the flow is returned to S510, and the processing is repeated.

As described above, the image sensor 1032 is disposed on the probe for treatment and diagnosis 1003, and the fluorescence acquired by the image sensor 1032 from the oral cavity and emitted to the excited light irradiated is mapped gradationally for the intensity at the controller and analyzer 1054, whereby the disinfection status of the sites irradiated with the treatment and diagnosis light can be perceived in real-time.

Next, a fourth embodiment will be described.

### Fourth Embodiment

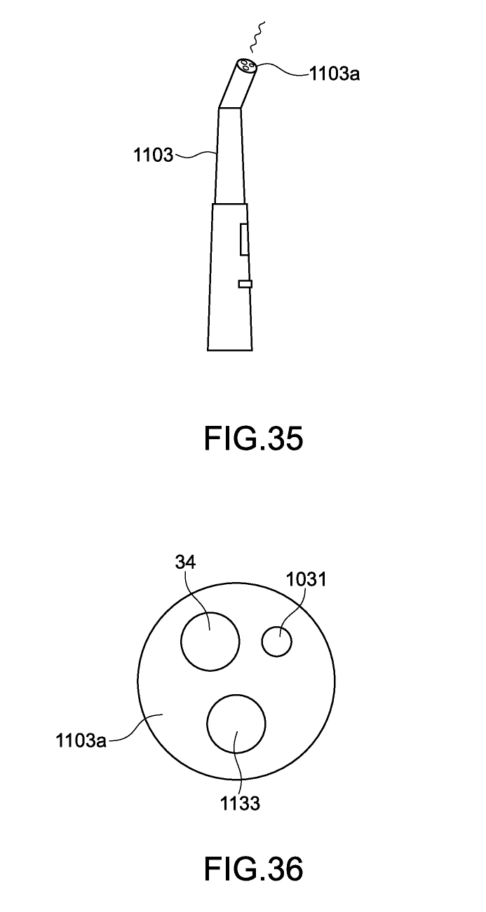

In the third embodiment, the probe for treatment and diagnosis 1003 is a lateral irradiation type, but may be a forward irradiation type. Hereinafter, the fourth embodiment will be described referring to FIGS. 35 to 37.

The configurations similar to the third embodiment are denoted by the same reference numerals, and thus detailed description thereof will be hereinafter omitted.

Each of the probes for treatment and diagnosis used in the first embodiment and the alternative embodiment is a forward irradiation type.

FIG. 35 is an overall view of the probe for treatment and diagnosis 1103.

FIG. 36 shows a tip surface of the probe for treatment and diagnosis 1103.

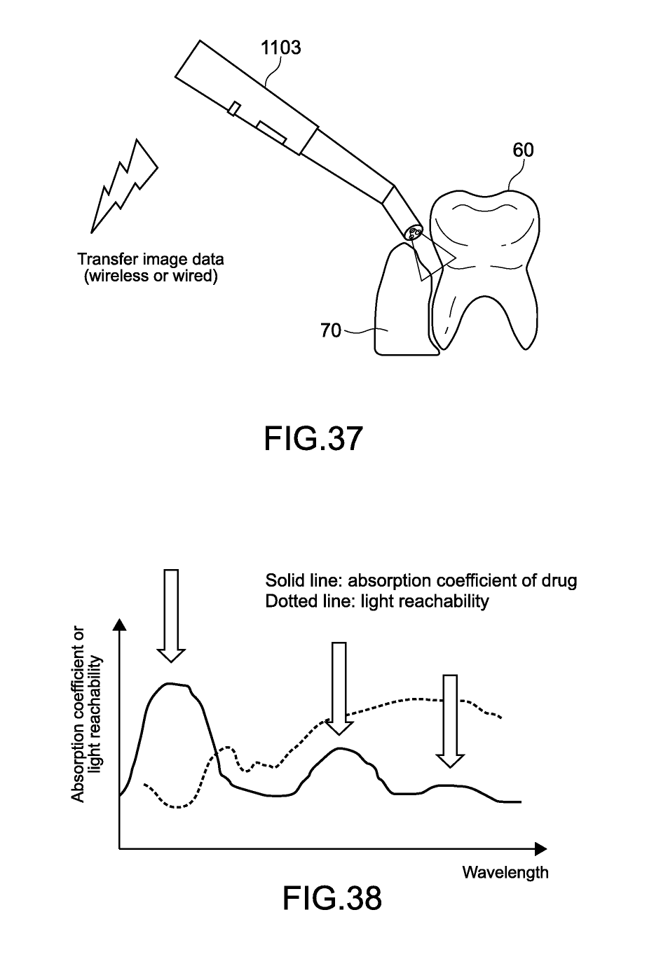

FIG. 37 shows treatment by the probe for treatment and diagnosis 1103.

A tip surface 1103a of the probe for treatment and diagnosis 1103 includes the probe for treatment and diagnosis 34, a light-receiving probe 1133 and an air outlet 1031.

The light-receiving probe 1133 includes the BPF and the photodiode. The BPF passes only the fluorescence included in the light incident on the light-receiving probe 1133 from the gum 70 side. The fluorescence passed is received by the photodiode.

As shown in FIG. 37, the periodontal pocket formed between the gum 70 and the tooth 60 is irradiated with the light emitted from the light irradiation probe for treatment and diagnosis 34. Next, a fifth embodiment will be described.

### Fifth Embodiment

In the above-described embodiments, the photosensitizer and the excited light are used to observe the disinfection of the periodontitis bacteria and the disinfection status. In contrast, autofluorescence of the plaques and the calculi attached to the gums and the teeth may be used to observe the removal status of the plaques and the calculi without using the photosensitizer.

In the fifth embodiment, when scaling is done using, for example, a hand scaler, a ultrasonic scaler or an air scaler, the oral cavity is irradiated with a blue light. A temporal change in a light emission intensity from the plaques and the calculi by irradiating the blue light is acquired in the similar way to the above-described embodiments for acquiring the temporal change in the fluorescence intensity. Alternatively, a fluorescence receiving means is disposed on a toothbrush instead of the scaler, and the temporal change in the light emission intensity from the plaques and the calculi by irradiating the blue light may be acquired.

For example, the scaler may include an irradiation unit for emitting the blue light to the treatment site, a light receiving unit that receives the light emission from the plaques and the calculi, and a control unit for outputting first data for visualizing the temporal change in the light intensity received by the light receiving unit.

In this way, the removal status of plaques and calculi can be observed in real time.

Although in the above-described third to fifth embodiments, the Doppler meter and the oxygen saturation meter for measuring the blood flow volume are not disposed, these may be disposed as in the first and second embodiments.

### General Description of Medical Apparatus

The above-described dental apparatus is used for periodontitis treatment and plaques and calculi removal. Medical apparatuses according to following embodiments can be used for treatment or prevention of infectious diseases in arthritis, laparoscopic surgery, choledocholithiasis, ptyalolithiasis, tooth root extraction and the like. Hereinafter, the medical apparatuses will be described. Configurations and operations similar to the above-described embodiments will be omitted or simplified.

In the aPDT, it is known that pathogenic bacteria such as periodontitis bacteria are disinfected by a photochemical reaction. It is also shown that the aPDT is widely effective for disinfecting infectious microorganisms such as viruses, protozoa, and fungi. The term “disinfection” herein is not limited to killing bacteria, and involves killing the infectious microorganisms.

Advantages of the aPDT include the following points:

Namely, it is considered that a pathogen will not show tolerance even if it is used repeatedly unlike antibiotics. Also, a diseased site of a patient can be preserved. In addition, the photosensitizer, the treated site of the patient and cell surfaces of bacteria are rapidly bonded by their electrostatic interactions to shorten the time from drug administration to treatment completion. From these advantages, the aPDT is expected as an infection treatment method alternative to antibiotics.

Furthermore, it is pointed that the aPDT has an immunostimulatory action (see Masamitsu Tanaka, Pawel Mroz, Tianhong Dail, Manabu Kinoshita, Yuji Morimoto and Michael R. Hamblin, “Photodynamic therapy can induce non-specific protective immunity against a bacterial infection” Proceedings of SPIE Vol. 8224 822403-1). In other words, it is shown that, by irradiating the site to which the photosensitizer is bonded with the excited light, a blood flow is improved, and neutrophils that are immune cells migrate to the site. Thus, the aPDT attracts an attention not only for infection treatment, but also for infection prevention.

In the following embodiments, a treatment progress such as the disinfection progress of the infectious microorganisms can be monitored based on the temporal change in the fluorescence intensity emitted from the irradiated site similar to the above-described embodiments. Thus, the aPDT can be effectively done. It may contribute to shortening of the treatment time and relapse prevention.

Hereinafter, each embodiment of the medical apparatus will be described.

### Sixth Embodiment

As the sixth embodiment, the medical apparatus for use in the prevention and treatment of arthritis will be described.

In recent years, the arthritis surgery is typically implemented by arthroscopic surgery.

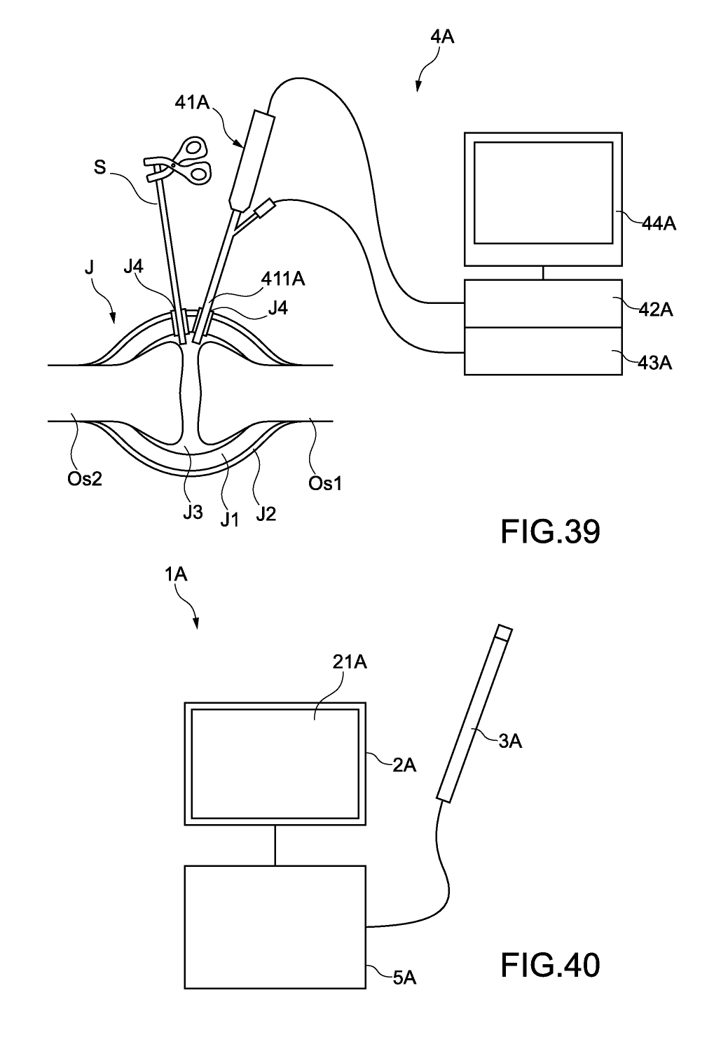

FIG. 39 is a schematic diagram showing the arthroscopic surgery.

A joint J has a joint capsule J1 that covers tip portions including cartilages of bones Os1 and Os2, a synovium J2 that configures an inner surface of the joint capsule J1, and a joint cavity J3 containing a synovial fluid covered by the synovium J2.

An arthroscope device 4A includes an arthroscope 41A having an insertion part 411A, a main unit 42A and a light source 43A connected to the arthroscope 41A, and a monitor 44A.

The arthroscope 41A is a hard mirror.

Under the arthroscopic surgery, some insertion holes J4 are formed through the joint capsule J1 and the synovium J2. Into the insertion holes J4, the insertion part 411A of the arthroscope 41A and a surgical instrument S such as forceps are inserted. On the monitor 44A, the image captured by the arthroscope 41A is displayed. The practitioner implements the surgery using the surgical instrument S, while visually recognizing the image.

Among arthritis, arthritis such as suppurative arthritis caused by bacteria infection via the blood flow and tuberculosis arthritis caused by tuberculosis bacteria injection can be treated by the aPDT. However, less blood flows through the joints, and an immune system is less activated. In other words, under the arthroscopic surgery of rheumatoid arthritis and gouty arthritis not caused by the infection, the bacteria infection will pose a high risk. Accordingly, the aPDT is effective for prevention of the infection before and after the arthroscopic surgery.

In the sixth embodiment, a preventive aPDT is implemented before the arthroscopic surgery, and a preventive or therapeutic aPDT is implemented during or after the surgery. In the aPDT according to the sixth embodiment, the synovium J2 including relatively many blood vessels and an immune system that is easily activated is used as the treatment site where at least one of the treatment and the prevention of the infectious diseases is implemented.

Hereinafter, the configuration of the medical apparatuses will be described.

### 1. Configuration of Medial Apparatus

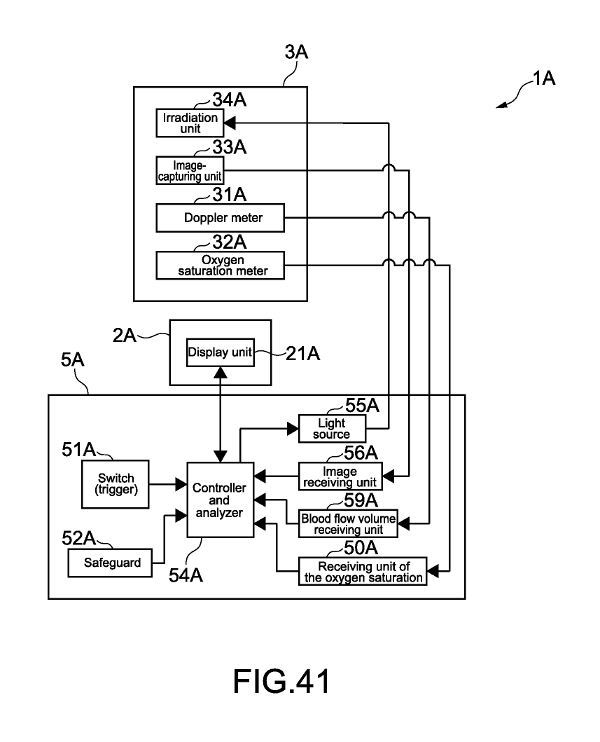

FIG. 40 shows a schematic configuration diagram of a medical apparatus 1A according to the sixth embodiment. FIG. 41 is a functional block diagram of the medical apparatus 1A shown in FIG. 40.

As shown in FIGS. 40 and 41, the medical apparatus 1A includes a monitor 2A, the main unit 5A, and a treatment probe 3A. In the sixth embodiment, based on a decreased amount (a bleaching amount) of the photosensitizer in the treatment site, a temporal change in, for example, a disinfection state at the site is graphed and displayed. The treatment probe 3A corresponds to “the probe for treatment and diagnosis” in the above-described embodiments.

### 1.1. Configuration of Monitor

The monitor 2A is a display apparatus having a display unit 21A displaying an image. The monitor 2A is connected wired or wireless to the main unit 5 similar to the above-described embodiments. Alternatively, the monitor 2A may not be disposed, and the main unit 5 may include the display unit. Also, a monitor 44A of the arthroscope device 4A may use used.

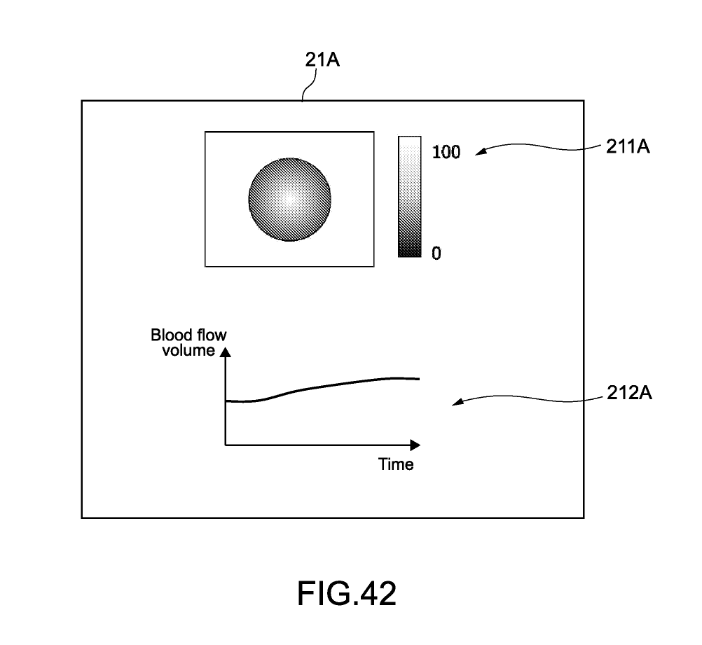

FIG. 42 shows an example of an image displayed on the display unit 21A of the monitor 2A.

At an upper area in the display unit 21A, an image 211A is displayed. In the image 211A, the temporal change in the fluorescence intensity at the treatment site is visualized.

At a lower area in the display unit 21A, a graph image 212A is displayed. The graph image 212A shows the temporal change in the blood flow volume measured by a Doppler meter 31A of the treatment probe 3A.

On the display unit 21A, an actual image (a camera image) of the treatment site captured by the treatment probe 3A, a graph of the oxygen saturation visualized by numerical values, as described later, and the like may be displayed.

### 1.2. Configuration of Treatment Probe

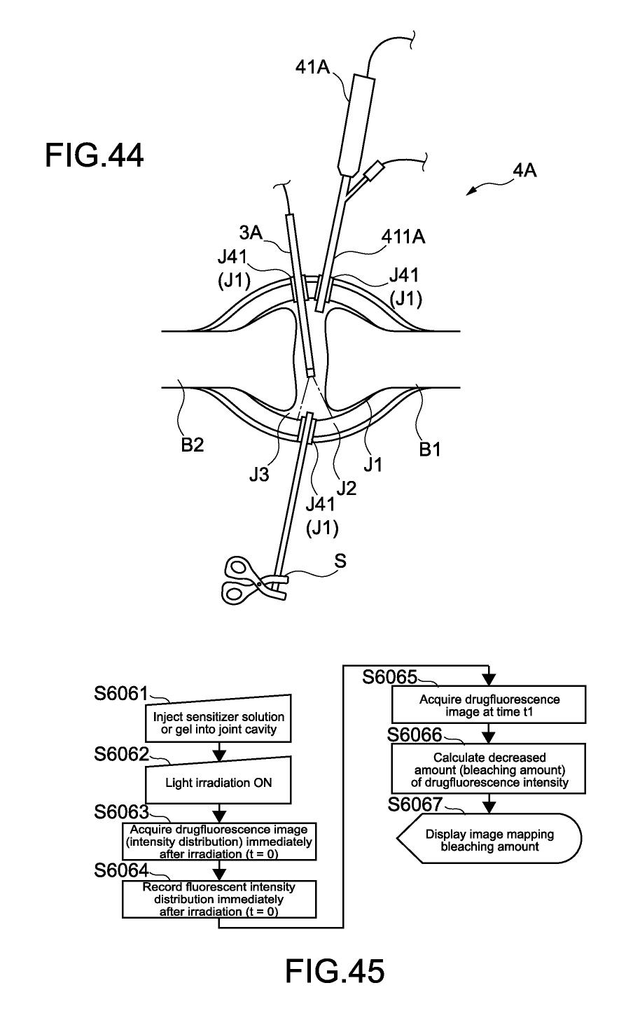

The treatment probe 3A is a stick having a gripper. The treatment probe 3A has the schematic configuration similar to that of the probe for treatment and diagnosis 1103 according to the fourth embodiment. Referring to FIGS. 41, 35, 36 etc., the treatment probe 3A includes a irradiation unit 34A, a light-receiving probe 33A, the Doppler meter 31A, and an oxygen saturation meter 32A. The treatment probe 3A according to the sixth embodiment is a forward irradiation type, and the synovium that is the treatment site is irradiated with the excitation light from a position distant from the synovium, for example (see FIG. 44).

The treatment probe 3A has a size that can pass through the insertion hole J4 formed under the arthroscopic surgery, and has a diameter of about 1 to 2 mm, for example.

The irradiation unit 34A includes fibers guiding a laser light and an LED light for treatment, having the wavelength belonging to the absorption band of the photosensitizer, emitted from the light source 55A of the main unit 5A as described later. The laser light and the LED light emitted from the light source 55A guided by the fibers are emitted forward of the irradiation unit 34A. The irradiated light emitted from the irradiation unit 34A may sterilize the bacteria bonded to the photosensitizer distributed in the synovium, or, as the prevention of the infectious diseases, may activate the immune system of the synovium itself bonded to the photosensitizer.

The light-receiving probe 33A converts the fluorescence image data emitted from the synovium into an electrical signal, and transmits the resultant image data to an image receiving unit 56A of the main unit 5A. The light-receiving probe 33A according to the sixth embodiment may include the BPF and the photodiode, as described in the fourth embodiment.

When disposing or blocking of the BPF can be switched on a light guide path of the light-receiving probe 33A, an actual image of the treatment site not irradiated can be acquired as well as the fluorescence image data, and can be displayed on the display unit 21A.

The Doppler meter 31A measures the blood flow volume of blood vessels at the treatment site with which the light for treatment and diagnosis is irradiated, for example, using the light having a wavelength of 633 nm. The blood flow volume is calculated using the Doppler shift as described above. Also, the blood flow volume may be determined by Fourier transformation of a speckle pattern of the reflected light.

The Doppler meter 31A transmits the information about the blood flow volume measured to a blood flow volume receiving unit 59A of the main unit 5A.

The oxygen saturation meter 32A measures oxygen saturation at the treatment site with which the irradiated light is irradiated, and has the configuration similar to the oxygen saturation meter 32 according to the first embodiment. In other words, by the oxygen saturation, a degree of inflammation in the arthritis can be quantitatively evaluated.

An oxygen saturation receiving unit 50A of the main unit 5A receives the oxygen saturation measured in the oxygen saturation meter 32A.

### 1.3. Configuration of Main Unit

As shown in FIG. 41, the main unit 5A includes the light source 55A, the image receiving unit 56A, and a controller and analyzer 54A. Also, the main unit 5A includes a switch 51A, a safeguard 52A, the blood flow volume receiving unit 59A, and the oxygen saturation receiving unit 50A.

The light source 55A emits a light (excited light) having the wavelength belonging to the absorption band of the photosensitizer administered into the treatment site.

The image receiving unit 56A is configured as a light detector that detects fluorescence from the treatment site emitted to a light irradiated from the light source 55A via the irradiation unit 34A. In other words, the image receiving unit 56A receives the fluorescence image data of the synovium etc. transmitted from the light-receiving probe 33A of the treatment probe 3A.

The blood flow volume receiving unit 59A receives the blood flow volume information measured by the Doppler meter 31A of the treatment probe 3A.

The oxygen saturation receiving unit 50A receives the oxygen saturation information measured by the oxygen saturation meter 32A of the treatment probe 3A.

The controller and analyzer 54A is configured as a control unit for outputting data for visualizing the temporal change in the fluorescence intensity based on the fluorescence detected by the image receiving unit 56A.

In other words, the controller and analyzer 54A gradationally maps the fluorescence intensity from the fluorescence image data acquired at the image-capturing unit 33A, outputs the data for visualizing the temporal change in the fluorescence intensity shown in FIG. 28D to the display unit 21A, and displays the image for visualizing the temporal change in the fluorescence intensity at the upper area on the display unit 21A.

The controller and analyzer 54A receives the blood flow volume information from the blood flow volume receiving unit 59A, outputs data for graphing and visualizing the relationship between the blood flow volume and the laser light irradiation time, and displays it at the lower area on the display unit 21A, as shown in FIG. 42.

The controller and analyzer 54A may receive the oxygen saturation information from the oxygen saturation receiving unit 50A, output data for numerically visualizing the oxygen saturation, and display it on the display unit 21A.

### 2. Photosensitizer

The photosensitizer is administered into the joint cavity, and is distributed over the synovium that is the treatment site.

The photosensitizer may be, but not limited to, the cation formulation such as methylene blue bonded to the bacteria by an electrostatic interaction, a solution or a gel of a drug taken into the bacteria similar to the first embodiment. Other photosensitizer such as porfimer sodium (Photofrin™), Talaporfin, 5-ALA and Foscan can be used. In the sixth embodiment, the practitioner locally administers the photosensitizer to the patient through the insertion hole J4 (see FIG. 39) using a DDS device such as an injection and a microneedle array.

### 3. Flow of Diagnosis and Treatment

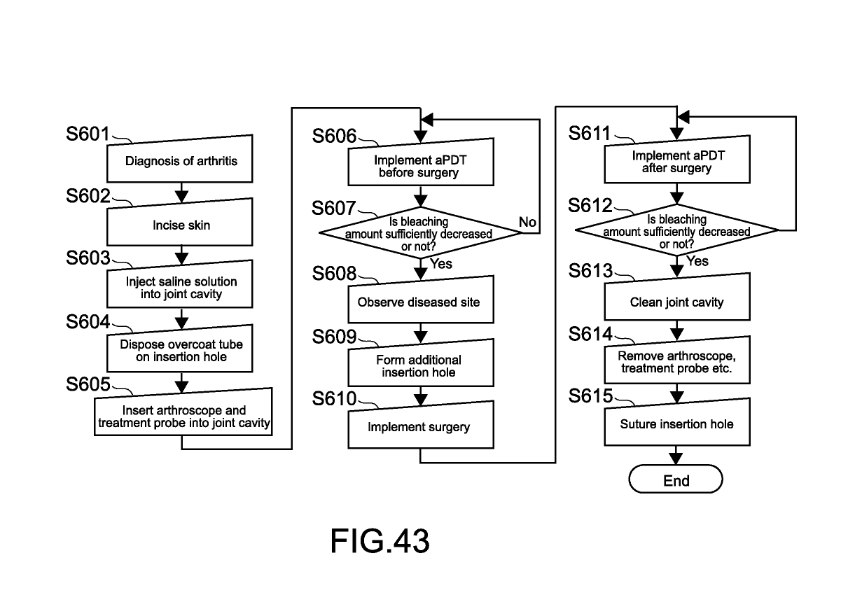

FIG. 43 shows a flow diagram showing a flow of diagnosis and treatment using the above-described medical apparatus 1A. FIG. 44 shows that the aPDT is implemented using the medical apparatus 1A. Referring to FIGS. 43 and 44, the flow of the diagnosis and treatment will be described.

### Diagnosis of Arthritis, S601

Prior to the surgery, the doctor etc. does a diagnosis about arthritis (S601). Specifically, the diagnosis of the arthritis is done by performing an MRI to the patient, interviewing the patient about a clinical condition by the doctor, etc. During the step, the patient diagnosed with the arthritis is explained by the doctor, and undergoes the arthroscopic surgery.

### Preparation of Arthroscopic Surgery, S602 to S605

After the patient transported to an operating room undergoes predetermined procedures including anesthesia administration, the practitioner incises a skin of a joint (S602). In this way, two or three small incisions (not shown) are formed on the skin. The small incision has a diameter of about 6 mm, for example.

Next, through the small incisions, a saline solution is injected to the joint cavity J3 (S603). Alternatively, carbon dioxide gas may be injected instead of the saline solution. In this way, the joint cavity J3 has an expanded volume for ease of surgery. Also, the saline solution may be perfused by a cannula etc. during the surgery to minimize an infection risk.

Each overcoat tubes J41 combined with a trocar (with a needle) is inserted into the small incisions. Then, the trocar perforates the joint capsule J1 and the synovium J2 to form the insertion holes J4 through the joint capsule J1 and the synovium J2 and to dispose the overcoat tubes J41 on the insertion holes J4 (S604). Thereafter, the trocar is removed. The overcoat tube J41 is a path for inserting the arthroscope 41A and the surgical instrument S from the insertion hole J4, and has a function to hermetically keep the insertion hole J4 such that the saline solution and the like will not leak outside. The trocar is not shown.

Then, the arthroscope 41A and the treatment probe 3A of the medical apparatus 1A are inserted into the joint cavity J3 through the insertion holes J4 (S605). Thus, tips of the arthroscope 41A and the treatment probe 3A are disposed within the joint cavity J3.

In the sixth embodiment, the preventive aPDT before the surgery is implemented prior to the actual surgery. In this way, the immune system of the treatment site, i.e., the synovium J2, is activated, and can decrease the infection risk. The image acquiring processing of the aPDT according to the sixth embodiment can be implemented similar to the third embodiment, which is described referring to FIGS. 28A to 28D showing the mapping of the treatment effect (the disinfection effect).

### Implementing aPDT Before Surgery, S606 to S607

Next, the aPDT before surgery is implemented (S606).

FIG. 45 is a flow diagram showing a flow of steps of implementing the aPDT before the surgery. The respective steps S6061 to S6067 included in the implementation of the aPDT surgery (S606) are shown.

Firstly, in order to administer the photosensitizer to the synovium J2, the photosensitizer is injected into the joint cavity J3 (S6061). In this way, the photosensitizer is distributed to the synovium J2.

Then, when the practitioner turns on the switch for controlling the irradiation of the light from irradiation unit 34A (S6062), the excited light is emitted from irradiation unit 34A. The excited light is irradiated to the synovium J2, and is diffused and reflected on the surface of the synovium J2.

Next, fluorescence acquisition processing is performed (S6063 to S6065).

The CMOS or CCD image sensor having the BPF of the treatment probe 3A acquires fluorescence data from the surface of the synovium J2 emitted to the exited light, and transmits the fluorescence data to the image receiving unit 56A of the main unit 5A.

The image receiving unit 56A acquires the drugfluorescence image (fluorescence intensity distribution) immediately after the irradiation (t=0) (S6063, see FIG. 28A). The image receiving unit 56A transmits the fluorescence intensity information at the time t=0 to the controller and analyzer 54A.

The controller and analyzer 54A records the distribution of the fluorescence intensity on the surface of the synovium J2 immediately after the irradiation (t=0) (S6064).

The image receiving unit 56A acquires the drugfluorescence image (fluorescence intensity distribution) at the time t=t1 (S6065, see FIG. 28B). The image receiving unit 56A transmits the fluorescence intensity information at the time t=t1 to the controller and analyzer 54A.

Then, the fluorescence intensity is calculated (S6066 to S6067).

The controller and analyzer 54A calculates a decreased amount (a bleaching amount) of the drugfluorescence intensity from the fluorescence intensity information at the time t=t0 and the fluorescence intensity information at the time t=t1 (S6066).

The controller and analyzer 54A outputs data for visualizing the bleaching amount during the time t1 based on the calculated bleaching amount, and displays the images as shown in FIG. 28C or 28D on the display unit 21 (S6067).

As described above, the practitioner implements the aPDT referring to the temporal change in the bleaching amount displayed on the display unit 21A.

The practitioner determines that the bleaching amount is sufficiently decreased or not based on the image displayed on the display unit 21A (S607). If it is determined to be sufficient (YES), the light irradiation is turned off.

If it is determined to be insufficient (NO), the processing is returned to S606, and the aPDT is continued.

Next, the arthroscopic surgery is implemented.

### Implementation of Arthroscopic Surgery, S608 to S610

The practitioner observes the diseased site, i.e., the joint hole J3, by the arthroscope 41A (S608). Depending on the status of the joint hole J3 observed, an additional insertion hole J4 may be further formed (S609).

Then, the surgery is implemented using the surgical instrument S (S610). In this way, a loose body of the joint cartilage is removed, or the diseased site is cut, as appropriate.

### Implementation of aPDT after Surgery, S611 to S612

The aPDT is implemented after the surgery for the disinfection treatment of the infectious arthritis, or for the prevention of the infectious disease of the non-infectious arthritis (S611). As this step is similar to those of the aPDT before the surgery (S6061 to S6067), the detailed description is omitted.

If the aPDT is implemented as the treatment, the data for visualizing the bleached amount of the photosensitizer is referred to as the data for visualizing the disinfection progress.

Also, if the aPDT is implemented as the treatment, the aPDT may be implemented during the surgery, or may be implemented multiple times during the surgery or after the surgery.

The practitioner determines that the bleaching amount is sufficiently decreased or not based on the image displayed on the display unit 21A as in the step S607 (S612). If it is determined to be sufficient (YES), the light irradiation is turned off.

If it is determined to be insufficient (NO), the processing is returned to S611, and the processing is repeated.

### Completion of Surgery, S613 to S615

Then, the practitioner cleans the joint cavity J3 with a saline solution and the like (S613). This step may be performed before the step S611. When the saline solution is perfused as described above, this step may not be performed.

The arthroscope 41A and the surgical instrument S are removed from the insertion holes J4 (S614), the overcoat tubes J41 are further removed, and the insertion holes J4 are sutured (S615).

As described above, according to the sixth embodiment, it is possible to prevent and treat effectively and rapidly the infectious diseases in the arthritis using the medical apparatus 1A. In this way, it is possible to prevent the infectious arthritis caused by the bacteria infection during the surgery from relapsing and developing, and inhibit a risk of developing multiple-drug-resistant bacteria. Also, it is possible to decrease patient's burden to take antibiotics.

### Alternative Embodiment

In the sixth embodiment, the excited light is irradiated using the light source 55A and the treatment probe 3A separated from the arthroscope 41A. Alternatively, the arthroscope may also be used as the treatment probe (the irradiation unit and the image-capturing unit).

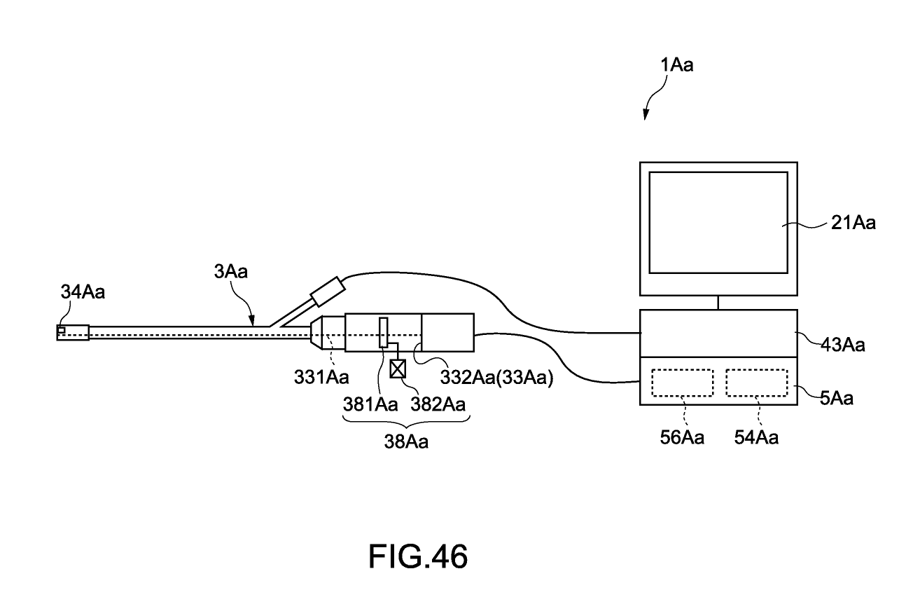

FIG. 46 is a schematic sectional diagram showing a configuration of the medical apparatus 1Aa according to an alternative embodiment of the sixth embodiment. The medical apparatus 1Aa is configured as the anthroscope. The medical apparatus 1Aa includes a light source 34Aa, an image-capturing unit 33Aa, an arthroscope 3Aa, a display unit 21Aa and a main unit 5Aa.

The light source 34Aa is, for example, an LED (light-emitting diode) disposed at a tip of the arthroscope 3Aa, and emits a light having the wavelength belonging to the absorption band of the photosensitizer.

The medical apparatus 1Aa may have a light source 43Aa for providing the irradiated light to acquire the actual image. The light source 43Aa is different from the light source 34Aa, and is connected to the arthroscope 3Aa.

The image-capturing unit 33Aa has a light receiving path 331Aa, on which optical systems (not shown) are disposed, and a photodiode 332Aa such as a CCD image sensor, and acquires the fluorescence image and the actual image at the treatment site.

The display unit 21Aa can display the actual image acquired by the image-capturing unit 33Aa, the image where the temporal change in the fluorescence intensity is visualized (see 211A in FIG. 42) and the like.

The main unit 5Aa includes the controller and analyzer 54Aa and an image receiving unit 56Aa. The image receiving unit 56Aa is configured as the light detector for detecting fluorescence from the treatment site emitted to the light irradiated from the light source 34Aa, and receives fluorescence image data or actual image data of the synovium transmitted from the image-capturing unit 33Aa. The controller and analyzer 54Aa outputs at least one of data for visualizing the temporal change in the fluorescence intensity based on the fluorescence detected by the image receiving unit 56Aa and the actual image data acquired.

By the above-described configuration, it is possible to irradiate the excited light from the arthroscope 41Aa, even if the arthroscope 41Aa has a small diameter, and fibers (an irradiation probe) for guiding the excited light for the photosensitizer cannot be disposed in addition to the fibers for guiding the excited light for acquiring the actual image.

The medical apparatus 1Aa may have a switch 38Aa for switching a mode where the image-capturing unit 33Aa acquires the fluorescence image from the treatment site and a mode where the image-capturing unit 33Aa acquires the actual image.

The switch 38Aa includes an optical filter 381Aa that can be disposed at the light-receiving path 331Aa, and a mechanism 382Aa for disposing or evacuating the optical filter 381Aa within the light receiving path 331Aa.

The optical filter 381Aa can limit the wavelength of the light to be transmitted to the wavelength belonging to the absorption band of the photosensitizer, and is configured of the BPF, for example.

The mechanism 382Aa may be connected to the controller and analyzer 54Aa, and may be drive-controlled by the controller and analyzer 54Aa. In this way, the mechanism 382Aa can be automatically driven. Alternatively, the mechanism 382Aa may be configured such that the position of the optical filter 381Aa can be manually switched. In this case, the mechanism 382Aa can be configured to have an insert formed on the arthroscope 3Aa and a guide for guiding the insertion of the optical filter 381Aa from the insert into the light receiving path 331Aa, thereby attaching and detaching the optical filter 381Aa.

In the mode where the image-capturing unit 33Aa acquires the actual image, the mechanism 382Aa evacuates the optical filter 381Aa from the light receiving path 331Aa. Thus, the medical apparatus 1Aa can be used as the arthroscope for acquiring and displaying the actual image of the diseased site. On the other hand, in the mode where the image-capturing unit 33Aa acquires the fluorescence image from the treatment site, the optical filer 381Aa is disposed on the light receiving path 331Aa.

Thus, the medical apparatus 1Aa can be used as the apparatus for treating or preventing the infectious diseases referring to the temporal change in the bleaching amount of the photosensitizer.

By the medical apparatus 1Aa, the number of the instruments used for the arthroscopic surgery can be decreased. Therefore, increasing the number of the insertion holes for the treatment probe is not necessary, leading to the lower-invasive surgery. In addition, changing a forceps etc. with the treatment probe in one insertion hole is not necessary. It is thus possible to further decrease the infection risk from the insertion hole.

The irradiation unit may have a diffuser for diffusing the emitted light.

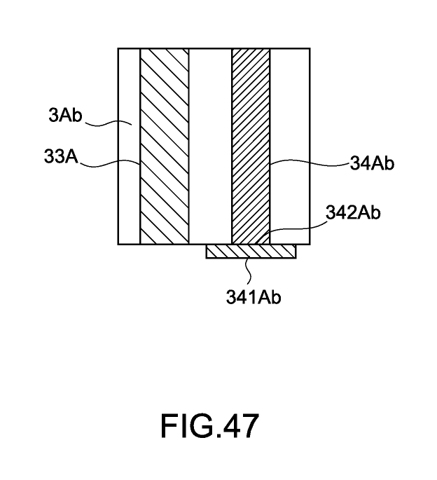

FIG. 47 is a schematic sectional diagram of the tip of the treatment probe 3Ab according to the alternative embodiment of the sixth embodiment. As the diffuser, a light diffusion plate 341Ab is disposed at an emission outlet 342Ab where the light is emitted from the fibers of the irradiation unit 34Ab. In this way, the emitted light is diffused, and a wider area of the synovium can be irradiated with the light. It is thus possible to improve the disinfection effect and immunostimulatory in the synovium.

Furthermore, the above-described treatment probe 3A includes the irradiation unit 34A of the fibers capable of guiding the light. Alternatively, the treatment probe 3A may include a lateral irradiation and needle-shaped irradiation unit as in the third embodiment.

Also, the image for visualizing the temporal change in the fluorescence intensity displayed on the above-described display unit 21A is not limited to the mapping of the fluorescence intensity as shown in FIG. 42, and may be a graph where an abscissa axis represents a time, and an ordinate axis represents the fluorescence intensity or the bleaching amount. This allows the practitioner to confirm the temporal change in the fluorescence intensity.

Alternatively, the medical apparatus may have a configuration capable of acquiring the three-dimensional model in the joints similar to the dental apparatus according to the first embodiment.

In addition, the above-described medical apparatus 1A has the configuration that includes the Doppler meter 31A and the oxygen saturation meter 32A, but may not include the both or one of them.

The medical apparatus may further include the above-described air blowing unit (see FIG. 26).

### Seventh Embodiment

As the seventh embodiment, the medical apparatus for use in the prevention and treatment of the laparoscopic surgery will be described.

In recent years, the laparoscopic surgery using a laparoscope is widely implemented in the fields of gynecology, urology and the like.

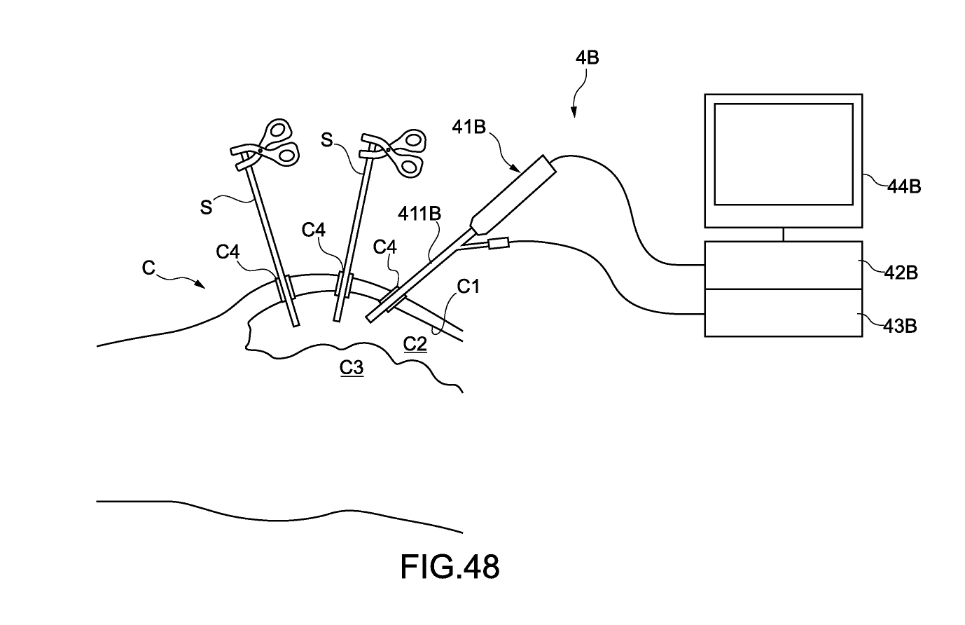

FIG. 48 is a schematic diagram showing a laparoscopic surgery.

An abdominal cavity C2 is a body cavity surrounded by a peritoneum C1, a diaphragm (not shown) or the like. Within the abdominal cavity C2, a plurality of organs C3 such as digestive organs, urinary organs and genital organs is disposed.

A laparoscope apparatus 4B includes a laparoscope 41B having an insertion part 411B, a main unit 42B and a light source 43B connected to the laparoscope 41B, and a monitor 44B.

The laparoscope 41B is a hard mirror similar to the arthroscope.

Under the laparoscopic surgery, some insertion holes C4 are formed through the peritoneum C1. Into each insertion hole C4, the insertion part 411B of the laparoscope 41B and a surgical instrument S such as forceps are inserted. On the monitor 44B, the image captured by the laparoscope 41B is displayed. The practitioner implements the surgery using the surgical instrument S, while visually recognizing the image.

Also, under the laparoscopic surgery, the bacteria infection will pose a high risk. At present, injection and removal of the saline solution is repeated two or three times to clean the abdominal cavity before the completion of the surgery, thereby cleaning the abdominal cavity to control the infection risks. However, it is difficult to deny an infection possibility by the operation.

Also, under the laparoscope surgery, it is possible to further decrease the injection risk by implementing the preventive aPDT. Some diseases to which the laparoscope surgery is applied may accompany the infectious diseases. In this case, the aPDT is implemented as the treatment to disinfect the bacteria, thereby decreasing the risk of reinfection.

In the seventh embodiment, the preventive aPDT is implemented before the laparoscopic surgery, and the preventive or therapeutic aPDT is implemented after the surgery, similar to the sixth embodiment. The treatment site in the aPDT according to the seventh embodiment can be the abdominal cavity C2. The “abdominal cavity” as the treatment site indicates the abdominal cavity C2 filled with the saline solution etc., the peritoneum around the insertion holes C4, the organ C3 diseased by the infection, etc. and can be appropriately set depending on a status of the disease to which the laparoscope surgery is applied.

The schematic configurations of the medical apparatus 1B according to the seventh embodiment are similar to the medical apparatus 1A according to the sixth embodiment are denoted by the same reference numerals, and thus detailed description thereof will be hereinafter omitted.

The treatment probe 3A according to the seventh embodiment is configured to have a size such that the treatment probe 3A can pass through the insertion hole C4 used in the laparoscope surgery, and has a diameter of about 3 to 10 mm, for example.

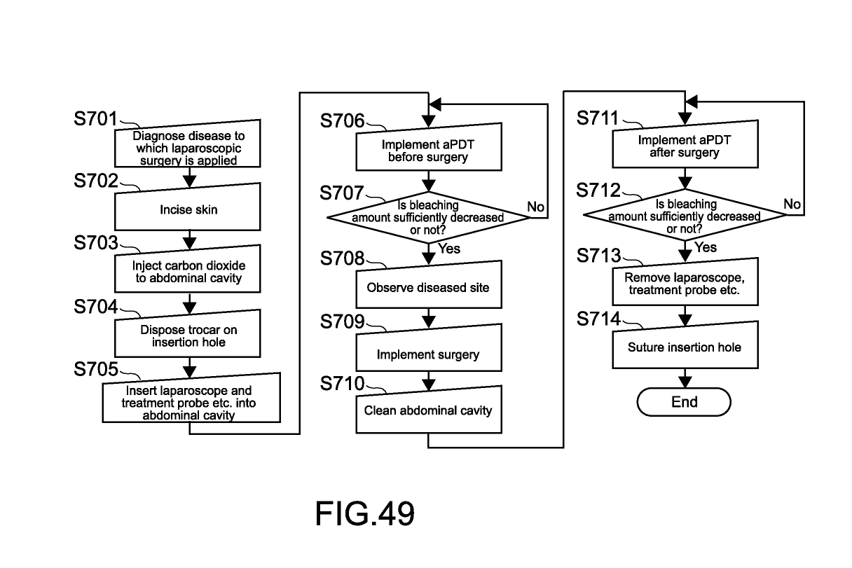

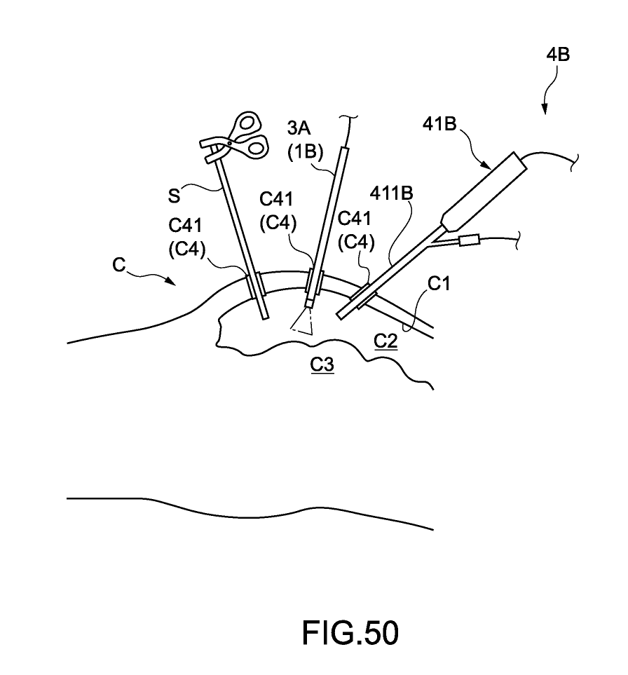

FIG. 49 shows a flow diagram showing a flow of diagnosis and treatment using the above-described medical apparatus 1B. FIG. 50 shows that the aPDT is implemented using the medical apparatus 1B. Referring to FIGS. 49 and 50, the flow of the diagnosis and treatment will be described.

### Diagnosis, S701

Prior to the surgery, the disease to which the laparoscope surgery is applied is diagnosed (S701). Specifically, extensive testing including a radiographic inspection, an ultrasonic inspection, a blood test and the like are made to the patient. In this step, when the doctor diagnoses the disease and decides that the laparoscopic surgery is necessary, the patient is explained by the doctor and undergoes the laparoscopic surgery.

### Preparation of Laparoscopic Surgery, S702 to S705

After the patient transported to an operating room undergoes predetermined procedures including anesthesia administration, the practitioner incises a skin of an abdomen (S702). In this way, a plurality of small incisions (not shown) is formed on the skin. The small incision has a diameter of about 5 to 12 mm, for example.

Next, through the small incisions, carbon dioxide is injected to the abdominal cavity C2 (S703). In this way, the abdominal cavity C2 has an expanded volume for ease of surgery. Also, the saline solution may be injected instead of carbon dioxide, or the saline solution may be perfused during the surgery.

Each overcoat tube (trocar) C41 is inserted into the small incisions. Then, the insertion hole C4 passing through the muscles of the abdomen or the peritoneum is formed, and the trocar C41 is disposed on the insertion hole C4 (S704). The trocar C41 is a path for inserting the laparoscope 41B and the surgical instrument S from the insertion hole C4, and has a function to hermetically keep the insertion hole C4 such that the carbon dioxide and the like will not leak outside.

Then, the laparoscope 41B and the treatment probe 3A of the medical apparatus 1B are inserted into the insertion hole C41 (S705). Thus, tips of the laparoscope 41B and the treatment probe 3A are disposed within the abdominal cavity C2.

### Implementing aPDT Before Surgery, S706 to S707

Then, the preventive aPDT before the surgery is implemented prior to the actual surgery (S706). In this way, the immune system around the treatment site is activated, and can decrease the infection risk. This step is similar to the aPDT before the surgery according to the sixth embodiment (S606), and thus detailed description thereof will be hereinafter omitted.

The practitioner determines that the bleaching amount is sufficiently decreased or not based on the image displayed on the display unit 21A (S707). If it is determined to be sufficient (YES), the light irradiation is turned off.

If it is determined to be insufficient (NO), the processing is returned to S706, and the aPDT is continued.

Next, the laparoscopic surgery is implemented.

### Implementation of Laparoscopic Surgery, S708 to S710

The practitioner observes the diseased site within the abdominal cavity C2 by the laparoscope 41B (S708). Depending on the observation result, the surgery is implemented using the surgical instrument S (S709). In this way, the diseased site is isolated, as appropriate.

Then, the practitioner cleans the abdominal cavity C2 with a saline solution and the like (S710). This step may be performed after the step S711. When the saline solution is perfused as described above, this step may not be performed.

### Implementation of aPDT after Surgery, S711 to S712

The aPDT is implemented after the surgery for the disinfection treatment of the infectious diseases, or for the infectious disease prevention of the non-infectious diseases (S711). As this step is similar to that of the aPDT before the surgery (S6061 to S6067), the detailed description is omitted.

If the aPDT is implemented as the treatment, the data for visualizing the bleached amount of the photosensitizer is referred to as the data for visualizing the disinfection progress.

Also, if the aPDT is implemented as the treatment, the aPDT may be implemented during the surgery, or may be implemented multiple times during the surgery or after the surgery.

The practitioner determines that the bleaching amount is sufficiently decreased or not based on the image displayed on the display unit 21A as in the step S707 (S712). If it is determined to be sufficient (YES), the light irradiation is turned off.

If it is determined to be insufficient (NO), the processing is returned to S711, and the processing is repeated.

### Completion of Surgery, S713 to S714

Then, the practitioner removes the laparoscope 41B, the treatment probe 3A and the surgical instrument S are removed from the insertion holes C4 (S713), and the insertion holes C4 are sutured (S714).

As described above, according to the seventh embodiment, it is possible to prevent and treat effectively and rapidly the infectious diseases under the laparoscopic surgery using the medical apparatus 1B. In this way, it is possible to prevent the infection caused by the bacteria infection during the surgery from relapsing and developing, and inhibit a risk of developing multiple-drug-resistant bacteria. Also, it is possible to decrease patient's burden to take antibiotics.

### Alternative Embodiment

In the seventh embodiment, the excited light is irradiated by inserting the treatment probe 3A from the insertion hole C4. Alternatively, the excited light may irradiate around the insertion hole C4 outside thereof before the insertion hole C4 is sutured. In this way, the insertion hole C4 having the highest infection risk can be effectively disinfected and prevented from the infected, and the infection risk can be also controlled by inserting the treatment probe 3A into the abdominal cavity C2.

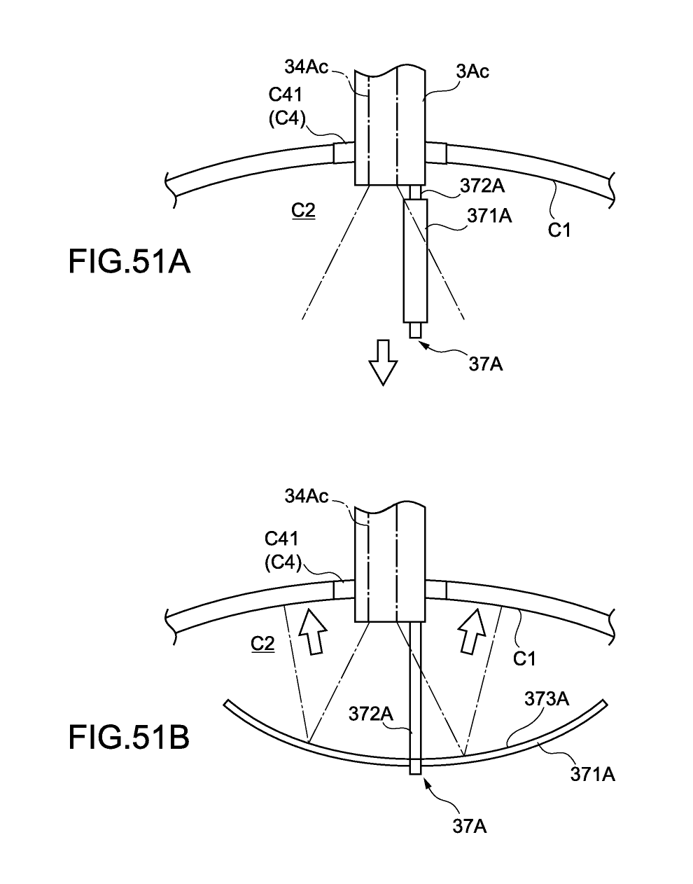

FIGS. 51A and 51B show that the aPDT is irradiated using the treatment probe 3Ac according to an alternative embodiment of the seventh embodiment. The medical apparatus 1B further includes a reflection unit 37A that reflects the excited light irradiated.

The reflection unit 37A has an umbrella configuration as a whole. In other words, the reflection unit 37A includes a cover member 371A capable of opening and closing freely and reflecting the excited light and a stick support member 372A supporting the cover member 371A and connected to the tip of the treatment probe 3Ac. Both members are disposed protrudedly from the tip of the treatment probe 3Ac. An inside surface of the cover member 371A forms a reflection plane 373A. The reflection plane 373A can reflect the light having the wavelength emitted from the irradiation unit 34Ac, for example.

FIG. 51A shows that the cover member 371A is closed. In this case, the reflection unit 37A is in a stick shape as a whole, similar to the closed umbrella, and the treatment probe 3Ac can irradiate the excited light from the irradiation unit 34Ac to a front direction.

On the other hand, FIG. 51B shows that the cover member 371A is opened. The reflection plane 373A is disposed facing to the peritoneum C1 around the insertion hole C4. In this way, the light emitted to the front direction is reflected on the reflection plane 373A, and can irradiate the peritoneum C1.

According to the alternative embodiment of the seventh embodiment, the excited light can effectively irradiate the peritoneum C1 around the insertion hole C4.

Accordingly, the peritoneum C1 around the insertion hole C4 having the high infection risk can be effectively disinfected, and the immune system in the peritoneum C1 can be activated.

The reflection unit 37A is not limited to the configuration that it is disposed at the treatment probe 3Ac. For example, the laparoscope 41B may have the reflection unit 37A that reflects the irradiated light from the treatment probe 3A. In this way, it is possible to adjust the position of the treatment probe 3A and the reflection unit 37A, thereby effectively reflecting the excited light.

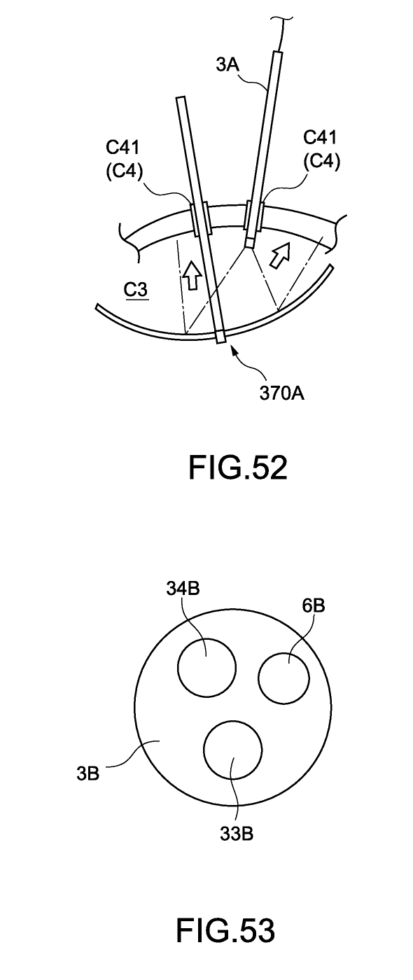

Furthermore, the reflection unit 37A may be configured as a separate reflection apparatus 370A as shown in FIG. 52. In this case, the reflection apparatus 370A can be disposable to be used more hygienically.

As shown in alternative embodiment of the sixth embodiment, the laparoscope may be also used as the treatment probe (see FIG. 46).

As the laparoscope has typically a larger diameter than the arthroscope, it is not limited to the configuration that the light source such as the LED is disposed at the tip. For example, similar to the sixth embodiment, the light source may be disposed at the main unit, and the irradiation probe such as fibers connected to the light source is disposed.

The medical apparatus 1B may further include a flow path 6B for a saline solution perfused within the abdominal cavity as a perfusion part.

FIG. 53 is a plan diagram showing a tip surface of the treatment probe 3B according to the alternative embodiment of the seventh embodiment. The treatment probe 3B includes an irradiation probe 34B, a CMOS or CCD image sensor or image fiber having a BPF 33B as the image-capturing unit, and the flow path 6B.

By the flow path 6B, it is possible to perfuse the saline solution within the abdominal cavity to further decrease the infection risk. In addition, without disposing a separate insertion hole for indwelling a cannula etc. for perfusion, the lower-invasive surgery can be implemented.

### Eighth Embodiment

In an eighth embodiment, a medical apparatus for use in treatment and relapse prevention of choledocholithiasis will be described.

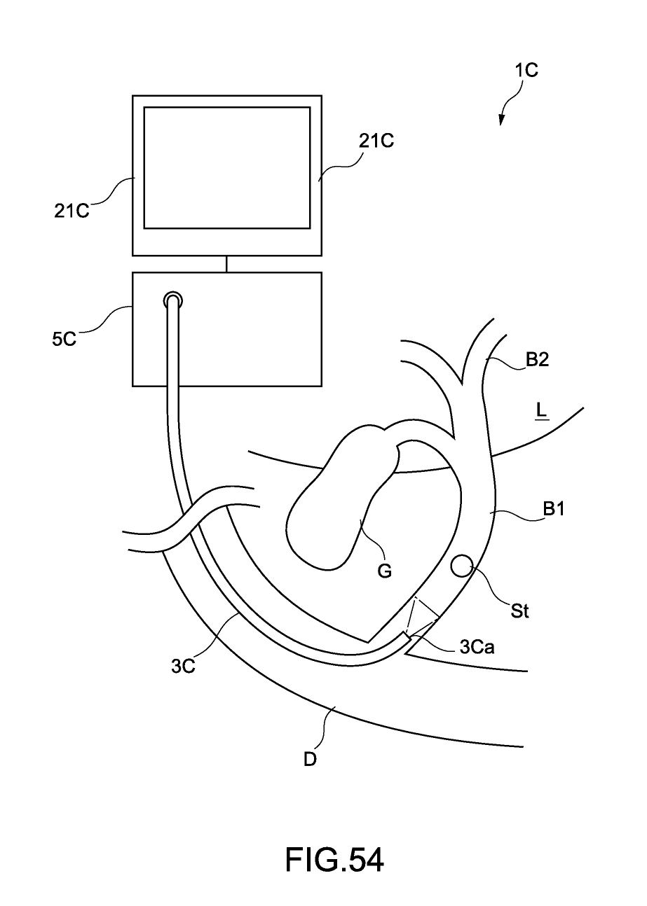

FIG. 54 is a schematic diagram showing the treatment of choledocholithiasis. Choledoch B1 has a tubular structure where a plurality of intrahepatic bile ducts B2 running internally and externally of a liver L is collected, and opens to duodenum D. The choledoch B1 is connected to a cholecyst G.

The choledocholithiasis is a disease that stone St clogs the choledoch B1. One of the cause is that gallbladder stone falls down to the choledoch B1 (fallen stone). Other cause is the bacteria infection within the choledoch B1. In other words, the bacteria such as Bacillus coli produce a mucosal fluid to form a bio film.

Bilirubin, calcium or the like is bonded thereto to form a primary stone.

The choledocholithiasis is treated using an endoscope. In other words, an endoscope 3C that is the soft mirror is inserted from the duodenum D, and a tip 3Ca is disposed around opening of the choledoch B1. In addition, a basket cannula (not shown) or the like is inserted into the choledoch B1, and the stone St within the choledoch B1 is removed.

Even though the bacteria infection is not related to the primary stone, a relapse risk is undeniable due to a second infection by the endoscopic treatment.

In the eighth embodiment, similar to the medical apparatus 1Aa according to the alternative embodiment of the sixth embodiment, a light source 34C including an LED and the like is disposed at the tip 3Ca of the endoscope 3C, and the choledoch B1 to which the photosensitizer is administered is irradiated with the excited light as the treatment site after the stone is removed. In this way, it is possible to implement the aPDT in order to prevent relapse of the choledocholithiasis.

Referring to FIG. 54, a medical apparatus 1C includes the endoscope 3C, a monitor 21C, a main unit 5C, an image-capturing unit 33C disposed at the endoscope 3C and a light source 34C. The endoscope 3C is a soft mirror as described above, and may have an operating part (not shown) or a connection part to the main unit 5C etc.

The configurations of the monitor 21C and the main unit 5C are similar to the metical apparatus 1Aa according to the alternative embodiment of the sixth embodiment, thus detailed description thereof is omitted, and the configuration of the endoscope 3C will be described.

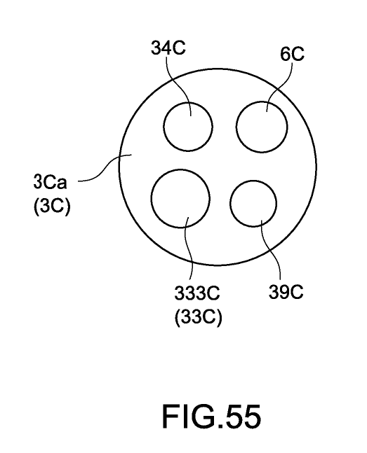

FIG. 55 is a plan view showing a configuration of the tip 3Ca of the endoscope 3C.

At the tip 3Ca, the light source 34C, an objective lens 333C, an irradiation probe for endoscope 39C and an insertion for forceps 6C are disposed.

The light source 34C is the LED similar to the light source 34Aa as shown in FIG. 46, and emits the light having the wavelength belonging to the absorption band of the photosensitizer. The light source 34C is also the irradiation unit.

The objective lens 333C is included in the image-capturing unit 33C, and disposed at a tip of a light-receiving path (not shown) of the image-capturing unit 33C.

The image-capturing unit 33C includes the objective lens 333C, the photodiode (not shown) (see FIG. 46), and the light-receiving path that guides the light to the photodiode. The image data acquired at the photodiode of the image-capturing unit 33C is transmitted to the image-receiving unit of the main unit 5C (see FIGS. 41 and 45).

An irradiation probe for endoscope 39C is used for illuminating the actual image upon the image-capturing by the endoscope 3C.

The insertion for forceps 6C has a hollow structure into which the surgical instrument such as the forceps is inserted. The insertion for forceps 6C may be a flow path for a liquid such as a saline solution or a gas.

An actual aPDT is described referring to FIG. 54.

After the stone is removed, the photosensitizer is administered to the choledoch B1 from the insertion for forceps 6C via a cannula and the like. The choledoch B1 is irradiated with the excited light emitted from the light source 34C.

The display unit 21C displays the image for visualizing the temporal change in the fluorescence intensity.

The practitioner refers to the image displayed on the display unit 21C, and determines that a decrease (a bleaching amount) of the fluorescence intensity is sufficient or not. If it is sufficient, the irradiation is terminated to end the surgery using the endoscope.

In this way, it is possible to effectively treat and prevent relapse of the choledocholithiasis.

Furthermore, the medical apparatus can be applied to other diseases as an alternative embodiment of the eighth embodiment.

### Alternative Embodiment about Other Diseases

One of the diseases is sialolithiasis.

The sialolithiasis is a disease that stones are formed in a salivary duct or a salivary gland. While the details of the cause is unclear, it is said that calcium contained in saliva is deposited around foreign matters or bacteria entered into the salivary duct.

A treatment method of the sialolithiasis includes image-capturing the diseased site using the hard mirror and removing the stones physically. Before and after the removal of the stones, the aPDT is implemented at the salivary duct or the salivary gland as the treatment site, whereby it is possible to disinfect and prevent relapse of the infected bacteria. In this case, the endoscope apparatus having the configuration similar to the above-described medical apparatus 1Aa can be used.

Another treatment method includes incising a floor of an oral cavity, and removing stones from a salivary duct outlet. Then, it is possible to implement the aPTD at an incision of the floor of the oral cavity after the removal of the stones. In this case, the medical apparatus having the configuration similar to the above-described medical apparatus 1A can be used.

The medical apparatus can be applied to a root canal treatment in the oral cavity.

The root canal is formed within a gum and is a canal housing tooth nerves. When the tooth nerves are infected with the bacteria by caries, the treatment for removing the nerves from the root canal is necessary. After the root canal treatment, the removal of a layer called a smeared layer attached to a root canal wall etc. is necessary. The smeared layer is formed by attaching cut dentin surface or tissue fragments to the root canal wall when the root canal is machinery cleaned by treatment instrument. In the smeared layer, the bacteria are likely to survive. At present, the smeared layer is removed by a cleaning liquid such as EDTA or steam foam.

In addition to the removal treatment of the smeared layer, the aPDT is implemented at the root canal as the treatment site before and after the removal of the smeared layer. In this way, it is possible to disinfect the root canal with certainty to contribute to the relapse prevention.

In this alternative embodiment, the medical apparatus having the configuration similar to, for example, the medical apparatus 1A according to the above-described sixth embodiment the can be used.

The diseases are not limited to the above-described ones, and the aPDT can be implemented at a diseased site of other infectious disease or a site being at risk of being infected as the treatment site using the above-described medical apparatuses.

Also, the above-described medical apparatus can be used for the aPDT to treat and prevent animal infection as well as human infection. Specifically, it is possible to apply the above-described medical apparatus to the treatment and relapse prevention of urolithiasis and the like.

The present technology may have the following configurations.

(1) A dental apparatus, including:

a light source for emitting a light to irradiate at least one of a tooth, a gum, a plaque and a calculus of an oral cavity;

a light detector for detecting fluorescence from the oral cavity emitted to the light irradiated from the light source; and

a control unit for outputting first data for visualizing a temporal change in a fluorescence intensity based on the fluorescence detected by the light detector.

(2) The dental apparatus according to (1) above, in which

a photosensitizer that is excited by irradiating the light is distributed in a depth direction of the gum such that the photosensitizer is bonded to or incorporated into periodontitis bacteria, and

the control unit outputs the first data based on a fluorescence intensity distribution in the depth direction of the gum from the photosensitizer emitted to the light irradiation.

(3) The dental apparatus according to (2) above, in which

the control unit calculates a temporal change in the fluorescence intensity in the depth direction based on a calculated temporal change in the distribution of the photosensitizer in a ground state in the depth direction, a calculated temporal change in an intensity distribution of the light in the depth direction, and a fluorescence intensity on a surface of the gum detected by the light detector.

(4) The dental apparatus according to (1) or (2) above, in which

the temporal change in the fluorescence intensity shows a disinfection progress of the periodontitis bacteria.

(5) The dental apparatus according to any one of (1) to (4) above, in which

a photosensitizer that is excited by irradiating the light is distributed in a depth direction of a plaque or a calculus attached to the tooth or the gum such that the photosensitizer is bonded to or incorporated into periodontitis bacteria, and

the control unit outputs the first data based on a fluorescence intensity distribution in the depth direction of the plaque or the calculus attached to the gum from the photosensitizer emitted to the light irradiation.

(6) The dental apparatus according to any one of (1) to (5) above, further including:

an image receiving unit for receiving an image of an oral cavity having the tooth and the gum;

a positional information receiving unit for receiving positional information about the oral cavity as absolute positional information from a reference position set on an arbitrary position; and

an image processing unit for linking image data received at the image receiving unit with positional information received at the positional information receiving unit, in which

the control unit correlates a light irradiated site of the oral cavity with the positional information, and outputs second data for showing the light irradiated site to the image of the oral cavity.

(7) The dental apparatus according to any one of (1) to (6) above, in which

a photosensitizer is administered into the oral cavity having the tooth and the gum, the photosensitizer being excited by the light irradiation and bonded to or incorporated into periodontitis bacteria, and

the control unit outputs the first data based on a fluorescence intensity distribution on a surface of the tooth or the gum from the photosensitizer around the surface of the tooth or the gum emitted to the light irradiation.

(8) The dental apparatus according to any one of (1) to (7) above, in which

the light is a laser light or a light-emitting diode light.

(9) The dental apparatus according to (1) above, in which

the light is a red light.

(10) The dental apparatus according to (9) above, in which

the light detector detects at least one of fluorescence, a reflected light and a diffused light from the oral cavity emitted to the red light.

(11) The dental apparatus according to any one of (1) to (10) above, further including:

a blood flow volume detector for detecting a blood flow volume of the gum.

(12) The dental apparatus according to any one of (1) to (11) above, further including:

an oxygen saturation meter for detecting oxygen saturation of the gum.

(13) The dental apparatus according to any one of (1) to (11) above, further including:

an air blowing unit for blowing air to the tooth or the gum.

(14) A calculation method including:

irradiating a gum of an oral cavity into which a photosensitize is administered with an excited light to the photosensitizer;

detecting a fluorescence intensity on a surface of the gum; and

calculating a temporal change in the fluorescence intensity in a depth direction based on a calculated temporal change in a distribution of the photosensitizer in the ground state to the depth direction of the gum, a calculated temporal change in the intensity distribution of the excited light in the depth direction, and the fluorescence intensity on the surface of the gum detected.

(15) A medical apparatus, including:

a light source for emitting a light to a treatment site where at least one of treatment and prevention of an infectious disease is implemented,

a light detector for detecting fluorescence from the treatment site emitted to the light irradiated from the light source; and

a control unit for outputting data for visualizing a temporal change in a fluorescence intensity based on the fluorescence detected by the light detector.

(16) The dental apparatus according to (15) above, in which

the treatment site is at least one of joint synovium, an abdominal cavity, a choledoch, a tooth root and a salivary gland.

(17) The dental apparatus according to (15) or (16) above, in which

the temporal change in the fluorescence intensity shows a disinfection progress of infectious microorganisms at the treatment site.

(18) The dental apparatus according to any one of (15) to (17) above, in which

a photosensitizer that is excited by irradiating the light irradiation is distributed to the treatment site, and

the control unit outputs the data based on a fluorescence intensity distribution at the treatment site from the photosensitizer emitted to the light irradiation.

(19) The dental apparatus according to any one of (15) to (18) above, further including:

a blood flow volume detector for detecting a blood flow volume of the treatment site.

(20) A calculation method including:

administering a photosensitizer to a treatment site where at least one of treatment and prevention of an infectious disease is implemented;

irradiating the treatment site with an excited light to the photosensitizer;

detecting a fluorescence intensity at the treatment site; and

calculating a temporal change in the fluorescence intensity at the treatment site based on the fluorescence intensity at the treatment site from the photosensitizer emitted to the light irradiation.

The present disclosure contains subject matter related to that disclosed in Japanese Priority Patent Applications JP 2012-125751 filed in the Japan Patent Office on Jun. 1, 2012, and Japanese Priority Patent Applications JP 2012-125751 and 2013-048767 filed in the Japan Patent Office on Mar. 12, 2013, the entire contents of which are hereby incorporated by reference.

It should be understood by those skilled in the art that various modifications, combinations, sub-combinations and alterations may occur depending on design requirements and other factors insofar as they are within the scope of the appended claims or the equivalents thereof.

What is claimed is:

1. A dental apparatus, comprising:
a light source for emitting a light to irradiate at least one of a tooth, a gum, a plaque and a calculus of an oral cavity;
a light detector for detecting fluorescence from the oral cavity emitted to the light irradiated from the light source; and
a control unit for outputting first data for visualizing a temporal change in a fluorescence intensity based on the fluorescence detected by the light detector.

2. The dental apparatus according to claim 1, wherein
a photosensitizer that is excited by irradiating the light is distributed in a depth direction of the gum such that the photosensitizer is bonded to or incorporated into periodontitis bacteria, and
the control unit outputs the first data based on a fluorescence intensity distribution in the depth direction of the gum from the photosensitizer emitted to the light irradiation.

3. The dental apparatus according to claim 2, wherein
the control unit calculates a temporal change in the fluorescence intensity in the depth direction based on a calculated temporal change in the distribution of the photosensitizer in a ground state in the depth direction, a calculated temporal change in an intensity distribution of the light in the depth direction, and a fluorescence intensity on a surface of the gum detected by the light detector.

4. The dental apparatus according to claim 3, wherein
the temporal change in the fluorescence intensity shows a disinfection progress of the periodontitis bacteria.

5. The dental apparatus according to claim 1, wherein
a photosensitizer that is excited by irradiating the light is distributed in a depth direction of one of a plaque and a calculus attached to one of the tooth and the gum such that the photosensitizer is bonded to or incorporated into periodontitis bacteria, and
the control unit outputs the first data based on a fluorescence intensity distribution in the depth direction of one of the plaque and the calculus attached to the gum from the photosensitizer emitted to the light irradiation.

6. The dental apparatus according to claim 1, further comprising:
an image receiving unit for receiving an image of an oral cavity having the tooth and the gum;
a positional information receiving unit for receiving positional information about the oral cavity as absolute positional information from a reference position set on an arbitrary position; and
an image processing unit for linking image data received at the image receiving unit with positional information received at the positional information receiving unit, wherein
the control unit correlates a light irradiated site of the oral cavity with the positional information, and outputs second data for showing the light irradiated site to the image of the oral cavity.

7. The dental apparatus according to claim 1, wherein
a photosensitizer is administered into the oral cavity having the tooth and the gum, the photosensitizer being excited by the light irradiation and bonded to or incorporated into periodontitis bacteria, and
the control unit outputs the first data based on a fluorescence intensity distribution on a surface of one of the tooth and the gum from the photosensitizer around the surface of one of the tooth and the gum emitted to the light irradiation.

8. The dental apparatus according to claim 1, wherein
the light is one of a laser light and a light-emitting diode light.

9. The dental apparatus according to claim 1, wherein
the light is a light having a wavelength belonging to an absorption band of the photosensitizer.

10. The dental apparatus according to claim 9, wherein
the light detector detects at least one of fluorescence, a reflected light and a diffused light from the oral cavity emitted to the light having the wavelength.

11. The dental apparatus according to claim 1, further comprising:
a blood flow volume detector for detecting a blood flow volume of the gum.

12. The dental apparatus according to claim 1, further comprising:
an oxygen saturation meter for detecting oxygen saturation of the gum.

13. The dental apparatus according to claim 1, further comprising:
an air blowing unit for blowing air to the tooth or the gum.

14. A calculation method comprising:
irradiating a gum of an oral cavity into which a photosensitize is administered with an excited light to the photosensitizer;
detecting a fluorescence intensity on a surface of the gum; and
calculating a temporal change in the fluorescence intensity in a depth direction based on a calculated temporal change in a distribution of the photosensitizer in the ground state to the depth direction of the gum, a calculated temporal change in the intensity distribution of the excited light in the depth direction, and the fluorescence intensity on the surface of the gum detected.

15. A medical apparatus, comprising:
a light source for emitting a light to a treatment site where at least one of treatment and prevention of an infectious disease is implemented,
a light detector for detecting fluorescence from the treatment site emitted to the light irradiated from the light source; and
a control unit for outputting data for visualizing a temporal change in a fluorescence intensity based on the fluorescence detected by the light detector.

16. The dental apparatus according to claim 15, wherein
the treatment site is at least one of joint synovium, an abdominal cavity, a choledoch, a tooth root and a salivary gland.

17. The dental apparatus according to claim 15, wherein
the temporal change in the fluorescence intensity shows a disinfection progress of infectious microorganisms at the treatment site.

18. The dental apparatus according to claim 15, wherein
a photosensitizer that is excited by irradiating the light irradiation is distributed to the treatment site, and
the control unit outputs the data based on a fluorescence intensity distribution at the treatment site from the photosensitizer emitted to the light irradiation.

19. The dental apparatus according to claim 15, further comprising:
a blood flow volume detector for detecting a blood flow volume of the treatment site.

20. A calculation method, comprising:
administering a photosensitizer to a treatment site where at least one of treatment and prevention of an infectious disease is implemented;
irradiating the treatment site with an excited light to the photosensitizer;
detecting a fluorescence intensity at the treatment site; and
calculating a temporal change in the fluorescence intensity at the treatment site based on the fluorescence intensity at the treatment site from the photosensitizer emitted to the light irradiation.
